SEQUENCE LISTING (43 sequences)

SEQ ID NO: 1 - protein, 147 aa, Homo sapiens
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENFRLLGNVLVCVLARNFGKEFTPQMQAAYQKVVAGVANALAHKYH

SEQ ID NO: 2 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 3 - protein, 467 aa, Homo sapiens [sequence omitted]

SEQ ID NO: 4 - protein, 1960 aa, Homo sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 5 - protein, 606 aa, Homo sapiens
MPYEIKKVFASLPQVERGVSKIIGGDPKGNNFLYTNGKCVILRNIDNPALADIYTEHAHQVVVAKYAPSGFYIASGDVSGKLRIWDTTQKEHLLKYEYQPFAGKIKDIAWTEDSKRIAVVGEGREKFGAVFLWDSGSSVGEITGHNKVINSVDIKQSRPYRLATGSDDNCAAFFEGPPFKFKFTIGDHSRFVNCVRFSPDGNRFATASADGQIYIYDGKTGEKVCALGGSKAHDGGIYAISWSPDSTHLLSASGDKTSKIWDVSVNSVVSTFPMGSTVLDQQLGCLWQKDHLLSVSLSGYINYLDRNNPSKPLHVIKGHSKSIQCLTVHKNGGKSYIYSGSHDGHINYWDSETGENDSFAGKGHTNQVSRMTVDESGQLISCSMDDTVRYTSLMLRDYSGQGVVKLDVQPKCVAVGPGGYAVVVCIGQIVLLKDQRKCFSIDNPGYEPEVVAVHPGGDTVAIGGVDGNVRLYSILGTTLKDEGKLLEAKGPVTDVAYSHDGAFLAVCDASKVVTVFSVADGYSENNVFYGHHAKIVCLAWSPDNEHFASGGMDMMVYVWTLSDPETRVKIQDAHRLHHVSSLAWLDEHTLVTTSHDASVKEWTITY

SEQ ID NO: 6 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 7 - protein, 397 aa, Homo sapiens
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEIDIRIRVPSIEFNANHPFLFFIRHNKTNTILFYGRLCSP

SEQ ID NO: 8 - protein, 431 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRGKHFISL

SEQ ID NO: 9 - protein, 95 aa, Homo sapiens
MTELETAMGMIIDVFSRYSGSEGSTQTLTKGELKVLMEKELPGFLQSGKDKDAVDKLLKDLDANGDAQVDFSEFIVFVAAITSACHKYFEKAGLK

SEQ ID NO: 10 - protein, 205 aa, Homo sapiens
MAEPLQPDPGAAEDAAAQAVETPGWKAPEDAGPQPGSYEIRHYGPAKWVSTSVESMDWDSAIQTGFTKLNSYIQGKNEKEMKIKMTAPVTSYVEPGSGPFSESTITISLYIPSEQQFDPPRPLESDVFIEDRAEMTVFVRSFDGFSSAQKNQEQLLTLASILREDGKVFDEKVYYTAGYNSPVKLLNRNNEVWLIQKNEPTKENE

SEQ ID NO: 11 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 12 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 13 - protein, 316 aa, Homo sapiens
MATFVELSTKAKMPIVGLGTWKSPLGKVKEAVKVAIDAGYRHIDCAYVYQNEHEVGEAIQEKIQEKAVKREDLFIVSKLWPTFFERPLVRKAFEKTLKDLKLSYLDVYLIHWPQGFKSGDDLFPKDDKGNAIGGKATFLDAWEAMEELVDEGLVKALGVSNFSHFQIEKLLNKPGLKYKPVTNQVECHPYLTQEKLIQYCHSKGITVTAYSPLGSPDRPWAKPEDPSLLEDPKIKEIAAKHKKTAAQVLIRFHIQRNVIVIPKSVTPARIVENIQVFDFKLSDEEMATILSFNRNWRACNVLQSSHLEDYPFNAEY

SEQ ID NO: 14 - protein, 901 aa, Homo sapiens
MEAARPSGSWNGALCRLLLLTLAILIFASDACKNVTLHVPSKLDAEKLVGRVNLKECFTAANLIHSSDPDFQILEDGSVYTTNTILLSSEKRSFTILLSNTENQEKKKIFVFLEHQTKVLKKRHTKEKVLRRAKRRWAPIPCSMLENSLGPFPLFLQQVQSDTAQNYTIYYSIRGPGVDQEPRNLFYVERDTGNLYCTRPVDREQYESFEIIAFATTPDGYTPELPLPLIIKIEDENDNYPIFTEETYTFTIFENCRVGTTVGQVCATDKDEPDTMHTRLKYSIIGQVPPSPTLFSMHPTTGVITTTSSQLDRELIDKYQLKIKVQDMDGQYFGLQTTSTCIINIDDVNDHLPTFTRTSYVTSVEENTVDVEILRVTVEDKDLVNTANWRANYTILKGNENGNFKIVTDAKTNEGVLCVVKPLNYEEKQQMILQIGVVNEAPFSREASPRSAMSTATVTVNVEDQDEGPECNPPIQTVRMKENAEVGTTSNGYKAYDPETRSSSGIRYKKLTDPTGWVTIDENTGSIKVFRSLDREAETIKNGIYNITVLASDQGGRTCTGTLGIILQDVNDNSPFIPKKTVIICKPTMSSAEIVAVDPDEPIHGPPFDFSLESSTSEVQRMWRLKAINDTAARLSYQNDPPFGSYVVPITVRDRLGMSSVTSLDVTLCDCITENDCTHRVDPRIGGGGVQLGKWAILAILLGIALLFCILFTLVCGASGTSKQPKVIPDDLAQQNLIVSNTEAPGDDKVYSANGFTTQTVGASAQGVCGTVGSGIKNGGQETIEMVKGGHQTSESCRGAGHHHTLDSCRGGHTEVDNCRYTYSEWHSFTQPRLGEKVYLCNQDENHKHAQDYVLTYNYEGRGSVAGSVGCCSERQEEDGLEFLDNLEPKFRTLAEACMKR

SEQ ID NO: 15 - protein, 256 aa, Homo sapiens
MFQTGGLIVFYGLLAQTMAQFGGLPVPLDQTLPLNVNPALPLSPTGLAGSLTNALSNGLLSGGLLGILENLPLLDILKPGGGTSGGLLGGLLGKVTSVIPGLNNIIDIKVTDPQLLELGLVQSPDGHRLYVTIPLGIKLQVNTPLVGASLLRLAVKLDITAEILAVRDKQERIHLVLGDCTHSPGSLQISLLDGLGPLPIQGLLDSLTGILNKVLPELVQGNVCPLVNEVLRGLDITLVHDIVNMLIHGLQFVIKV

SEQ ID NO: 16 - protein, 737 aa, Homo sapiens
MQPRRAQAPGAQLLPALALLLLLLGAGPRGSSLANPVPAAPLSAPGPCAAQPCRNGGVCTSRPEPDPQHPAPAGEPGYSCTCPAGISGANCQLVADPCASNPCHHGNCSSSSSSSSDGYLCICNEGYEGPNCEQALPSLPATGWTESMAPRQLQPVPATQEPDKILPRSQATVTLPTWQPKTGQKVVEMKWDQVEVIPDIACGNASSNSSAGGRLVSFEVPQNTSVKIRQDATASLILLWKVTATGFQQCSLIDGRSVTPLQASGGLVLLEEMLALGNNHFIGFVNDSVTKSIVALRLTLVVKVSTCVPGESHANDLECSGKGKCTTKPSEATFSCTCEEQYVGTFCEEYDACQRKPCQNNASCIDANEKQDGSNFTCVCLPGYTGELCQSKIDYCILDPCRNGATCISSLSGFTCQCPEGYFGSACEEKVDPCASSPCQNNGTCYVDGVHFTCNCSPGFTGPTCAQLIDFCALSPCAHGTCRSVGTSYKCLCDPGYHGLYCEEEYNECLSAPCLNAATCRDLVNGYECVCLAEYKGTHCELYKDPCANVSCLNGATCDSDGLNGTCICAPGFTGEECDIDINECDSNPCHHGGSCLDQPNGYNCHCPHGWVGANCEIHLQWKSGHMAESLTNMPRHSLYIIIGALCVAFILMLIILIVGICRISRIEYQGSSRPAYEEFYNCRSIDSEFSNAIASIRHARFGKKSRPAMYDVSPIAYEDYSPDDKPLVTLIKTKDL

SEQ ID NO: 17 - protein, 493 aa, Homo sapiens
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 18 - protein, 193 aa, Homo sapiens
MQSLMQAPLLIALGLLLAAPAQAHLKKPSQLSSFSWDNCDEGKDPAVIRSLTLEPDPIIVPGNVTLSVMGSTSVPLSSPLKVDLVLEKEVAGLWIKIPCTDYIGSCTFEHFCDVLDMLIPTGEPCPEPLRTYGLPCHCPFKEGTYSLPKSEFVVPDLELPSWLTTGNYRIESVLSSSGKRLGCIKIAASLKGI

SEQ ID NO: 19 - protein, 119 aa, Homo sapiens
MEFGLSWVFLVAILKGVQCEVQLVESGGGLVQPGRSLRLSCTASGFTFGDYAMSWVRQAPGKGLEWVGFIRSKAYGGTTEYAASVKGRFTISRDDSKSIAYLQMNSLKTEDTAVYYCTR

SEQ ID NO: 20 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 21 - protein, 249 aa, Homo sapiens
MLQLWKLVLLCGVLTGTSESLLDNLGNDLSNVVDKLEPVLHEGLETVDNTLKGILEKLKVDLGVLQKSSAWQLAKQKAQEAEKLLNNVISKLLPTNTDIFGLKISNSLILDVKAEPIDDGKGLNLSFPVTANVTVAGPIIGQIINLKASLDLLTAVTIETDPQTHQPVAVLGECASDPTSISLSLLDKHSQIINKFVNSVINTLKSTVSSLLQKEICPLIRIFIHSLDVNVIQQVVDNPQHKTQLQTLI

SEQ ID NO: 22 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 23 - protein, 501 aa, Homo sapiens
MQVCSQPQRGCVREQSAINTAPPSAHNAASPGGARGHRVPLTEACKDSRIGGMMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 24 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 25 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 26 - protein, 712 aa, Homo sapiens
MRVLLHLPALLASLILLQAAASTTRAQTTRTSAISDTVSQAKVQVNKAFLDSRTRLKTAMSSETPTSRQLSEYLKHAKGRTRTAIRNGQVWEESLKRLRQKASLTNVTDPSLDLTSLSLEVGCGAPAPVVRCDPCSPYRTITGDCNNRRKPALGAANRALARWLPAEYEDGLSLPFGWTPGKTRNGFPLPLAREVSNKIVGYLNEEGVLDQNRSLLFMQWGQIVDHDLDFAPDTELGSSEYSKAQCDEYCIQGDNCFPIMFPPNDPKAGTQGKCMPFFRAGFVCPTPPYKSLAREQINALTSFLDASFVYSSEPSLASRLRNLSSPLGLMAVNQEVSDHGLPYLPYDSKKPSPCEFINTTARVPCFLAGDSRASEHILLATSHTLFLREHNRLARELKRLNPQWDGEKLYQEARKILGAFVQIITFRDYLPILLGDHMQKWIPPYQGYSESVDPRISNVFTFAFRFGHLEVPSSMFRLDENYQPWGPEPELPLHTLFFNTWRMVKDGGIDPLVRGLLAKKSKLMKQNKMMTGELRNKLFQPTHRIHGFDLAAINTQRCRDHGQPGYNSWRAFCDLSQPQTLEELNTVLKSKMLAKKLLGLYGTPDNIDIWIGAIAEPLVERGRVGPLLACLLGKQFQQIRDGDRFWWENPGVFTNEQKDSLQKMSFSRLVCDNTRITKVPRDPFWANSYPYDFVDCSAIDKLDLSPWASVKN

SEQ ID NO: 27 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 28 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 2413 aa, Homo sapiens
MGISTVILEMCLLWGQVLSTGGWIPRTTDYASLIPSEVPLDPTVAEGSPFPSESTLESTVAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALRLVNGGDRCQGRVEVLYRGSWGTVCDDSWDTNDANVVCRQLGCGWATSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWATSAPGNARFGQGSGPIVLDDVRCSGHESYLWSCPHNGWLSHNCGHHEDAGVICSVSQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQLGCGWAMSAPGNARFGQGSGPIVLDDVRCSGHESYLWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 30 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 31 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 32 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 33 - protein, 590 aa, Homo sapiens
MHLARLVGSCSLLLLLGALSGWAASDDPIEKVIEGINRGLSNAEREVGKALDGINSGITHAGREVEKVFNGLSNMGSHTGKELDKGVQGLNHGMDKVAHEINHGIGQAGKEAEKLGHGVNNAAGQVGKEADKLIHHGVHHGANQAGSEAGKFGQGVDNAAGQAGNEAGRFGQGVHHAAGQAGNEAGRFGQGVHHAAGQAGNEAGRFGQGAHHGLSEGWKETEKFGQGIHHAAGQVGKEAEKFGQGAHHAAGQAGNEAGRFGQGVHHGLSEGWKETEKFGQGVHHTAGQVGKEAEKFGQGAHHAAGQAGNEAGRFGQGAHHAAGQAGNEAGRFGQGVHHGLSEGWKETEKFGQGVHHAASQFGKETEKLGHGVHHGVNEAWKEAEKFGQGVHHAASQVGKEEDRVVQGLHHGVSQAGREAGQFGHDIHHTAGQAGKEGDIAVHGVQPGVHEAGKEAGQFGQGVHHTLEQAGKEADKAVQGFHTGVHQAGKEAEKLGQGVNHAADQAGKEVEKLGQGAHHAAGQAGKELQNAHNGVNQASKEANQLLNGNHQSGSSSHQGGATTTPLASGASVNTPFINLPALWRSVANIMP

SEQ ID NO: 34 - protein, 484 aa, Homo sapiens
MAGPWTFTLLCGLLAATLIQATLSPTAVLILGPKVIKEKLTQELKDHNATSILQQLPLLSAMREKPAGGIPVLGSLVNTVLKHIIWLKVITANILQLQVKPSANDQELLVKIPLDMVAGFNTPLVKTIVEFHMTTEAQATIRMDTSASGPTRLVLSDCATSHGSLRIQLLHKLSFLVNALAKQVMNLLVPSLPNLVKNQLCPVIEASFNGMYADLLQLVKVPISLSIDRLEFDLLYPAIKGDTIQLYLGAKLLDSQGKVTKWFNNSAASLTMPTLDNIPFSLIVSQDVVKAAVAAVLSPEEFMVLLDSVLPESAHRLKSSIGLINEKAADKLGSTQIVKILTQDTPEFFIDQGHAKVAQLIVLEVFPSSEALRPLFTLGIEASSEAQFYTKGDQLILNLNNISSDRIQLMNSGIGWFQPDVLKNIITEIIHSILLPNQNGKLRSGVPVSLVKALGFEAAESSLTKDALVLTPASLWKPSSPVSQ

SEQ ID NO: 35 - protein, 377 aa, Homo sapiens
MKTLPLFVCICALSACFSFSEGRERDHELRHRRHHHQSPKSHFELPHYPGLLAHQKPFIRKSYKCLHKRCRPKLPPSPNNPPKFPNPHQPPKHPDKNSSVVNPTLVATTQIPSVTFPSASTKITTLPNVTFLPQNATTISSRENVNTSSSVATLAPVNSPAPQDTTAAPPTPSATTPAPPSSSAPPETTAAPPTPSATTQAPPSSSAPPETTAAPPTPPATTPAPPSSSAPPETTAAPPTPSATTPAPLSSSAPPETTAVPPTPSATTLDPSSASAPPETTAAPPTPSATTPAPPSSPAPQETTAAPITTPNSSPTTLAPDTSETSAAPTHQTTTSVTTQTTTTKQPTSAPGQNKISRFLLYMKNLLNRIIDDMVEQ

SEQ ID NO: 36 - protein, 357 aa, Homo sapiens
MGRQLAGCGDAGKKASFKMSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 37 - protein, 313 aa, Homo sapiens
MRALVLLLSLFLLGGQAQHVSDWTYSEGALDEAHWPQHYPACGGQRQSPINLQRTKVRYNPSLKGLNMTGYETQAGEFPMVNNGHTVQISLPSTMRMTVADGTVYIAQQMHFHWGGASSEISGSEHTVDGIRHVIEIHIVHYNSKYKSYDIAQDAPDGLAVLAAFVEVKNYPENTYYSNFISHLANIKYPGQRTTLTGLDVQDMLPRNLQHYYTYHGSLTTPPCTENVHWFVLADFVKLSRTQVWKLENSLLDHRNKTIHNDYRRTQPLNHRVVESNFPNQGKGHGGHRGRSQNPRVQPTSTRHPLALGSLEA

SEQ ID NO: 38 - protein, 623 aa, Homo sapiens
MSCRQFSSSYLSRSGGGGGGGLGSGGSIRSSYSRFSSSGGGGGGGRFSSSSGYGGGSSRVCGRGGGGSFGYSYGGGSGGGFSASSLGGGFGGGSRGFGGASGGGYSSSGGFGGGFGGGSGGGFGGGYGSGFGGFGGFGGGAGGGDGGILTANEKSTMQELNSRLASYLDKVQALEEANNDLENKIQDWYDKKGPAAIQKNYSPYYNTIDDLKDQIVDLTVGNNKTLLDIDNTRMTLDDFRIKFEMEQNLRQGVDADINGLRQVLDNLTMEKSDLEMQYETLQEELMALKKNHKEEMSQLTGQNSGDVNVEINVAPGKDLTKTLNDMRQEYEQLIAKNRKDIENQYETQITQIEHEVSSSGQEVQSSAKEVTQLRHGVQELEIELQSQLSKKAALEKSLEDTKNRYCGQLQMIQEQISNLEAQITDVRQEIECQNQEYSLLLSIKMRLEKEIETYHNLLEGGQEDFESSGAGKIGLGGRGGSGGSYGRGSRGGSGGSYGGGGSGGGYGGGSGSRGGSGGSYGGGSGSGGGSGGGYGGGSGGGHSGGSGGGHSGGSGGNYGGGSGSGGGSGGGYGGGSGSRGGSGGSHGGGSGFGGESGGSYGGGEEASGSGGGYGGGSGKSSHS

SEQ ID NO: 39 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 40 - protein, 117 aa, Homo sapiens
MAGFPLLLTLLTHCAGSWAQSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSG

SEQ ID NO: 41 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 42 - protein, 1049 aa, Homo sapiens
MDWSFFRVVAMLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 43 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

(19) United States
(12) Patent Application Publication
(10) Pub. No.: US 2021/0140979 A1
(43) Pub. Date: May 13, 2021

(54) METHOD OF DIAGNOSING PERIODONTAL CONDITIONS USING SALIVARY PROTEIN MARKERS

(71) Applicants: AJOU UNIVERSITY INDUSTRY -ACADEMIC COOPERATION FOUNDATION, Suwon-si, (KR); THE CATHOLIC UNIVERRSITY OF KOREA INDUSTRY-ACADEMIC COOPERATION FOUNDATION, Seoul (KR); SEOUL NATIONAL UNIVERSITY R&DB FOUNDATION, Seoul (KR); KOREA UNIVERSITY RESEARCH AND BUSINESS FOUNDATION, Seoul (KR)

(72) Inventors: Suk JI, Seoul (KR); Young Kyung KO, Seoul (KR); Joo Cheol PARK, Seoul (KR); Man Bock GU, Seoul (KR); Seong Min BAK, Yongin-si (KR); Eun Mi LEE, Anyang-si (KR); Yea Jin LEE, Seoul (KR); Geum Bit HWANG, Seoul (KR); Bang Hyun LEE, Seoul (KR)

(73) Assignees: AJOU UNIVERSITY INDUSTRY-ACADEMIC COOPERATION FOUNDATION, Suwon-si (KR); THE CATHOLIC UNIVERSITY OF KOREA INDUSTRY-ACADEMIC COOPERATION FOUNDATION, Seoul (KR); SEOUL NATIONAL UNIVERSITY R&DB FOUNDATION, Seoul (KR); KOREA UNIVERSITY RESEARCH AND BUSINESS FOUNDATION, Seoul (KR)

(21) Appl. No.: 17/095,178

(22) Filed: Nov. 11, 2020

(30) Foreign Application Priority Data
Nov. 12, 2019 (KR) 10-2019-0144047

(51) Int. Cl. G01N 33/68; G01N 33/72

(52) U.S. Cl. CPC G01N 33/6893; G01N 2800/18; G01N 33/721

(57) ABSTRACT

A method of diagnosing a periodontal condition using a protein showing a concentration difference between periodontal tissue in a normal condition and an abnormal condition from a subject's saliva is provided. According to this non-invasive method, the patient himself is able to check the presence of periodontal disease and identify the time to receive a treatment at an early stage, which will allow the patient to save time and cost for the treatment of periodontitis.

### CROSS-REFERENCE TO RELATED APPLICATION

This application claims the benefit of Korean Patent Application No. 10-2019-0144047, filed on Nov. 12, 2019 in the Korean Intellectual Property Office, the entire disclosure of which are incorporated herein by reference.

### BACKGROUND

### 1. Field of the Invention

The present invention relates to a biomarker for diagnosing a periodontal condition, and more particularly, to a method of diagnosing a periodontal condition using a protein showing an expression difference in oral saliva between a normal condition and an abnormal condition as a marker.

### 2. Discussion of Related Art

Periodontal diseases such as periodontitis are chronic diseases leading to the severe destruction of tissue around teeth due to inflammation that has developed without a patient's awareness. Therefore, it is common for patients to visit dentists late in an advanced stage requiring tooth extraction.

Today, a method of measuring a periodontal pocket depth by inserting a probe into the gingival sulcus, which is one of the diagnostic methods used to diagnose periodontitis, is a method for determining how much alveolar bone has been lost and confirming a degree of gingival inflammation, and the most basic diagnostic method for periodontitis. However, the method of measuring a periodontal pocket depth may have errors depending on the shape of a tooth and the degree of gingival inflammation.

A method of confirming bone loss on a radiograph is the most basic method of diagnosing periodontitis along with periodontal probing pocket depth. However, this method can show the loss of the alveolar bone on the mesial and distal surfaces of a tooth by a two-dimensional image, but has a limitation in that it may not show the loss of alveolar bone on the buccal and lingual surfaces of a tooth at which the tooth overlaps the image.

In addition, the method of confirming a periodontal pocket depth and bone loss on a radiograph shows only the result of alveolar bone loss (attachment loss) according to the progression of periodontitis before the time of diagnosis, and thus has a problem of not being able to show the active state of the current disease.

On the other hand, currently, the most effective method showing whether there is inflammation of the gingiva at the time of diagnosis is a method of checking for bleeding by inserting a probe into the sulcus between a tooth and the gingiva. However, this method has a limitation in that it is highly likely to show false positives (even when there is bleeding at the time of probing, the actual gingiva may not be inflamed).

Such conventional methods are methods conducted by experts in the clinic, and thus cumbersome and expensive, and depending on the method, a patient's pain necessarily accompanies.

Therefore, for periodontal disease in which symptoms appear late, it is necessary to develop a new method that can easily diagnose the current inflammatory condition of periodontal tissue or a healthy condition without inflammation.

That is, if the patient himself is able to check the presence or absence of periodontal disease and receives a treatment early, it will be possible to drastically reduce the time and cost associated with the treatment of periodontal diseases.

### RELATED PATENT DOCUMENTS

KR 1731764

US 2008/0027146

### SUMMARY OF THE INVENTION

The present invention is directed to providing a novel biomarker that is able to be used in diagnosing a periodontal condition.

The present invention is also directed to providing a simple method of diagnosing a periodontal condition, which is able to reduce a patient's effort and save time and money using a non-invasive method.

One aspect of the present invention provides, in order to provide information required for diagnosis of a periodontal condition, a method of detecting a marker protein for diagnosing a periodontal condition, which includes:

i) detecting one or more proteins selected from the group consisting of Hemoglobin subunit delta, Histone H3.1, Neutrophil collagenase, Myosin-9, WD repeat-containing protein 1, Cathepsin G, Serpin B10, Vimentin, Protein S100-P, Heme-binding protein 2, Alpha-actinin-4, Protein disulfide-isomerase, Ig lambda constant 2, Ig heavy constant alpha 2, BPI fold-containing family A member 2, Ig heavy constant mu, Lactoperoxidase, Glyceraldehyde-3-phosphate dehydrogenase, KRT6A Keratin, type II cytoskeletal 6A, Isoform 2 of Interleukin-1 receptor antagonist protein, BPI fold-containing family A member 1, Desmocollin-2, Phospholipid transfer protein, Aldo-keto reductase family 1 member B10, Isoform 2 of Clusterin, Leucine-rich alpha-2-glycoprotein, Deleted in malignant brain tumors 1 protein, Ig heavy variable 3-49, Ganglioside GM2 activator, Carcinoembryonic antigen-related cell adhesion molecule 6, Delta and Notch-like epidermal growth factor-related receptor, Phosphoglycerate kinase 1, Suprabasin, BPI fold-containing family B member 1, Mucin-7, Annexin A2, Carbonic anhydrase 6, Keratin, type I cytoskeletal 9, Alpha-1-antichymotrypsin, Ig lambda variable 1-47, Zinc-alpha-2-glycoprotein, Desmoglein-1 and Phosphatidylethanolamine-binding protein 1 from a subject's sample; and

ii) associating the subject with the diagnosis of a periodontal condition, if the above-listed one or more proteins are increased or decreased in concentration, in comparison with a control sample.

Another aspect of the present invention provides a composition for diagnosing a periodontal condition, which contains a reagent for detecting the above-listed one or proteins or an immunogenic fragment thereof.

In addition, still another aspect of the present invention provides a kit for diagnosing a periodontal condition, which includes a composition containing a reagent for detecting the above-listed one or more proteins or an immunogenic fragment thereof.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a Venn diagram showing the number of proteins detected in five samples per each group with three repeated experiments in a proteome analysis:

The number of proteins detected in common in 5 persons of a periodontitis patient group in all of three repeated experiments was 110, and among these proteins, 26 proteins (Table A) are proteins which were detected in all subjects of a periodontitis patient group in all experiments, but were not detected in all subjects of the periodontally healthy group or the group after periodontitis treatment in all of three repeated experiments;

the number of proteins detected in common in 5 persons of a periodontally healthy group in all three repeated experiments is 101, and among these proteins, 24 proteins (Table B) were detected in all five subjects of a periodontally healthy group in all experiments, but were not detected in subjects of the periodontitis patient group or the group after periodontitis treatment in three repeated experiments; and

the number of proteins detected in common in 5 persons of the periodontitis patient group in three repeated experiments was 109, and among these proteins, 11 proteins (Table C) were common proteins detected in common in 5 persons of the healthy group in all of three experiments.

FIG. 2 is an image showing the result of gel electrophoresis with saliva samples from 15 persons, which were separated according to molecular weight and stained with Coomassie Brilliant Blue R-250, followed by MS/MS analysis; and

FIGS. 3A to 3C show some of mass spectrometer images obtained from three saliva samples.

### DETAILED DESCRIPTION OF EXEMPLARY EMBODIMENTS

Hereinafter, the present invention will be described in further detail with reference to exemplary embodiments. These examples are merely provided to illustrate the present invention, and it should not be construed that the scope of the present invention is limited by the following embodiments.

Contents that are not described herein may be sufficiently technically inferred by those of ordinary skill in the art to which the present invention belongs, and therefore the descriptions thereof will be omitted.

The term “diagnosis” used herein refers to confirmation of the presence or absence of a disease or illness. Specifically, the diagnosis may mean determination of a periodontally healthy condition, a condition in which periodontal disease is suspected, or a condition in which periodontal disease or inflammation is resolved through treatment.

The term “diagnostic marker” used herein refers to a material that is able to determine a periodontally healthy condition, a periodontal condition in which periodontal disease or inflammation is resolved or an abnormal condition in which periodontal disease is suspected, and includes an organic biomolecule such as a protein, a polypeptide, a nucleic acid or a fragment thereof.

The term “detection” used herein refers to quantitative and/or qualitative analysis, and includes detection of the presence or absence, and detection of an existing amount (level).

The term “sample” used herein refers to a subject-derived specimen in which a marker protein of the present invention is present, and preferably, saliva non-invasively obtained from a subject.

The term “periodontal disease” used herein may include, but is not limited thereto, periodontitis and gingivitis.

The term “normal” used herein refers to a condition in which the gums are healthy or periodontal disease or inflammation is resolved by treatment, and the “abnormal” used herein refers to a condition in which the gums exhibit symptoms of a suspected disease such as periodontitis.

The “and/or” used herein means the preceding word (phrase), the trailing word (phrase), or both of the preceding word (phrase) and the trailing word (phrase).

The applicants confirmed that the expression of a protein listed in Table 1A increases in a sample of an individual with chronic periodontal disease, and the expression of proteins listed in Tables 1B, 2, 3 and 4 increases in samples of individuals which have healthy gums or in which inflammation is resolved after the treatment of periodontitis, and thus, confirmed these proteins can be used as protein markers that help in diagnosing a periodontal condition.

TABLE 1A

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| P02042 | Hemoglobin | 0.002 | 1.906 | 0.719 | 1 | 5 | 2 |
| | subunit delta | | | | | | |
| P68431 | Histone H3.1 | 0.007 | 0.130 | 0.082 | 1 | 5 | 4 |
| P22894 | Neutrophil | 0.008 | 0.024 | 0.019 | 4 | 5 | 5 |
| | collagenase | | | | | | |
| P35579 | Myosin-9 | 0.004 | 0.012 | 0.017 | 4 | 5 | 5 |
| O75083 | WD repeat- | 0.007 | 0.018 | 0.012 | 5 | 5 | 4 |
| | containing | | | | | | |
| | protein 1 | | | | | | |
| P08311 | Cathepsin G | 0.022 | 0.059 | 0.063 | 2 | 5 | 4 |
| P48595 | Serpin B10 | 0.008 | 0.020 | 0.018 | 4 | 5 | 4 |
| B0YJC4 | Vimentin | 0.011 | 0.027 | 0.018 | 2 | 5 | 4 |
| P25815 | Protein | 0.032 | 0.069 | 0.046 | 5 | 5 | 5 |
| | S100-P | | | | | | |

| | Detection count | | | | |
|---|---|---|---|---|---|
| | | Before | After | Ratio | |
| | Healthy | Treatment | Treatment | Before/ | GO Biological |
| Uniprot ID | group | group | group | Healthy | Function |
| P02042 | 1 | 15 | 5 | 956.388 | blood coagulation |
| P68431 | 2 | 15 | 11 | 18.571 | blood coagulation |
| P22894 | 10 | 15 | 12 | 2.962 | collagen catabolic |
| | | | | | process/neutrophil |
| | | | | | degranulation |
| P35579 | 11 | 15 | 14 | 2.845 | actin cytoskeleton |
| | | | | | reorganization/ |
| | | | | | leukocyte migration |
| O75083 | 13 | 15 | 12 | 2.701 | actin filament |
| | | | | | depolymerization/ |
| | | | | | neutrophil mediated |
| | | | | | immunity |
| P08311 | 6 | 15 | 12 | 2.695 | angiotensin |
| | | | | | maturation/ |
| | | | | | antibacterial |
| | | | | | humoral response |
| P48595 | 9 | 15 | 11 | 2.581 | negative |
| | | | | | regulation of |
| | | | | | endopeptidase |
| | | | | | activity/neutrophil |
| | | | | | degranulation |
| B0YJC4 | 5 | 15 | 7 | 2.408 | NA |
| P25815 | 13 | 15 | 14 | 2.159 | endothelial cell |
| | | | | | migration/neutrophil |
| | | | | | degranulation |

TABLE 1B

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| Q9Y5Z4 | Heme- | 0.026 | 0.013 | 0.012 | 5 | 5 | 5 |
| | binding | | | | | | |
| | protein 2 | | | | | | |
| O43707 | Alpha- | 0.102 | 0.047 | 0.064 | 5 | 5 | 5 |
| | actinin-4 | | | | | | |
| P07237 | Protein | 0.080 | 0.015 | 0.060 | 5 | 5 | 5 |
| | disulfide- | | | | | | |
| | isomerase | | | | | | |

| | Detection count | | | | |
|---|---|---|---|---|---|
| | | Before | After | Ratio | |
| | Healthy | Treatment | Treatment | Before/ | GO Biological |
| Uniprot ID | group | group | group | Healthy | Function |
| Q9Y5Z4 | 14 | 15 | 13 | 0.488 | negative regulation of |
| | | | | | mitochondrial membrane |
| | | | | | potential/neutrophil |
| | | | | | degranulation |
| O43707 | 14 | 15 | 14 | 0.460 | actin filament bundle |
| | | | | | assembly/platelet |
| | | | | | degranulation |
| P07237 | 14 | 15 | 13 | 0.191 | cell redox |
| | | | | | homeostasis/cellular |
| | | | | | response to |
| | | | | | IL7, 12, 23 |

TABLE 2

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| O60218 | Aldo-keto | 0.040 | 0.002 | 0.014 | 5 | 4 | 5 |
| | reductase family | | | | | | |
| | 1 member B10 | | | | | | |
| Q02487 | Desmocollin-2 | 0.058 | 0.007 | 0.026 | 5 | 5 | 5 |
| Q9NP55 | BPI fold- | 0.071 | 0.011 | 0.069 | 5 | 4 | 4 |
| | containing family | | | | | | |
| | A member 1 | | | | | | |
| Q8NFT8 | Delta and Notch- | 0.005 | 0.001 | 0.004 | 5 | 4 | 5 |
| | like epidermal | | | | | | |
| | growth factor- | | | | | | |
| | related receptor | | | | | | |
| P55058 | Phospholipid | 0.040 | 0.008 | 0.037 | 5 | 3 | 4 |
| | transfer protein | | | | | | |
| P17900 | Ganglioside GM2 | 0.029 | 0.007 | 0.011 | 5 | 5 | 4 |
| | activator | | | | | | |
| A0A0A0MS15 | Ig heavy variable | 0.031 | 0.008 | 0.013 | 5 | 5 | 5 |
| | 3-49 | | | | | | |
| P18510-2 | Isoform 2 of | 0.138 | 0.036 | 0.046 | 5 | 5 | 5 |
| | Interleukin-1 | | | | | | |
| | receptor | | | | | | |
| | antagonist | | | | | | |
| | protein | | | | | | |
| Q96DR5 | BPI fold- | 0.605 | 0.163 | 0.704 | 5 | 4 | 5 |
| | containing family | | | | | | |
| | A member 2 | | | | | | |
| P40199 | Carcinoembryonic | 0.020 | 0.006 | 0.012 | 5 | 5 | 5 |
| | antigen-related | | | | | | |
| | cell adhesion | | | | | | |
| | molecule 6 | | | | | | |
| P10909-2 | Isoform 2 of | 0.038 | 0.011 | 0.037 | 5 | 5 | 5 |
| | Clusterin | | | | | | |
| P02538 | KRT6A Keratin, | 0.199 | 0.061 | 0.114 | 5 | 5 | 5 |
| | type II | | | | | | |
| | cytoskeletal 6A | | | | | | |
| P02750 | Leucine-rich | 0.036 | 0.013 | 0.027 | 5 | 5 | 5 |
| | alpha-2- | | | | | | |
| | glycoprotein | | | | | | |
| P22079 | Lactoperoxidase | 0.209 | 0.084 | 0.205 | 5 | 5 | 5 |
| A0A0G2JMB2 | Ig heavy constant | 1.056 | 0.460 | 0.469 | 5 | 4 | 3 |
| | alpha 2 | | | | | | |
| | (Fragment) | | | | | | |
| P0DOY2 | Ig lambda | 4.221 | 1.850 | 3.218 | 5 | 5 | 5 |
| | constant 2 | | | | | | |
| Q9UGM3 | Deleted in | 0.036 | 0.016 | 0.026 | 5 | 5 | 5 |
| | malignant brain | | | | | | |
| | tumors 1 protein | | | | | | |
| P01871 | Ig heavy constant | 0.371 | 0.177 | 0.187 | 5 | 5 | 5 |
| | mu | | | | | | |
| P04406 | Glyceraldehyde- | 0.201 | 0.098 | 0.288 | 5 | 5 | 5 |
| | 3-phosphate | | | | | | |
| | dehydrogenase | | | | | | |

| | Detection count | | | | |
|---|---|---|---|---|---|
| | | Before | After | Ratio | |
| | Healthy | Treatment | Treatment | Healthy/ | GO Biological |
| Uniprot ID | group | group | group | Before | Function |
| O60218 | 15 | 6 | 13 | 18.095 | cellular detoxification of |
| | | | | | aldehyde/daunorubicin |
| | | | | | metabolic process/ |
| | | | | | doxorubicin metabolic |
| | | | | | process |
| Q02487 | 15 | 10 | 14 | 7.711 | cell adhesion/ |
| | | | | | keratinization |
| Q9NP55 | 15 | 11 | 10 | 6.552 | innate immune response |
| Q8NFT8 | 15 | 5 | 12 | 5.817 | Notch signaling pathway/ |
| | | | | | endocytosis/skeletal |
| | | | | | muscle fiber |
| | | | | | development |
| P55058 | 15 | 8 | 11 | 5.313 | ceramide transport/ |
| | | | | | lipid metabolic process |
| P17900 | 15 | 10 | 10 | 4.011 | neutrophil degranulation/ |
| | | | | | ganglioside catabolic |
| | | | | | process/ |
| | | | | | oligosaccharide |
| | | | | | catabolic process |
| A0A0A0MS15 | 15 | 7 | 12 | 3.821 | B cell receptor signaling |
| | | | | | pathway/innate |
| | | | | | immune response |
| P18510-2 | 15 | 13 | 13 | 3.781 | immune response |
| Q96DR5 | 15 | 11 | 14 | 3.708 | antimicrobial humoral |
| | | | | | response |
| P40199 | 15 | 11 | 14 | 3.412 | leukocyte migration/ |
| | | | | | apoptotic process/ |
| | | | | | neutrophil degranulation |
| P10909-2 | 15 | 12 | 14 | 3.404 | immune complex |
| | | | | | clearance/innate |
| | | | | | immune response/lipid |
| | | | | | metabolic process/ |
| | | | | | platelet degranulation |
| P02538 | 15 | 12 | 13 | 3.281 | antimicrobial humoral |
| | | | | | immune response/ |
| | | | | | keratinization/cell |
| | | | | | differentiation |
| P02750 | 15 | 14 | 13 | 2.862 | neutrophil degranulation/ |
| | | | | | positive regulation of |
| | | | | | endothelial cell |
| | | | | | proliferation |
| P22079 | 15 | 13 | 14 | 2.475 | cell redox homeostasis/ |
| | | | | | defense response to |
| | | | | | bacterium |
| A0A0G2JMB2 | 15 | 12 | 8 | 2.296 | NA |
| P0DOY2 | 15 | 12 | 12 | 2.282 | innate immune response |
| Q9UGM3 | 15 | 13 | 12 | 2.240 | innate immune response/ |
| | | | | | epithelial cell |
| | | | | | differentiation |
| P01871 | 15 | 14 | 14 | 2.100 | immune response |
| P04406 | 15 | 13 | 13 | 2.050 | canonical glycolysis/ |
| | | | | | microtubule |
| | | | | | cytoskeleton |
| | | | | | organization/ |
| | | | | | antimicrobial humoral |
| | | | | | immune response |
| | | | | | mediated by |
| | | | | | antimicrobial peptide |

TABLE 3

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| P00558 | Phosphoglycerate | 0.373 | 0.084 | 0.137 | 5 | 5 | 5 |
| | kinase 1 | | | | | | |
| Q6UWP8 | Suprabasin | 0.009 | 0.003 | 0.008 | 5 | 5 | 5 |
| Q8TDL5 | BPI fold- | 0.153 | 0.051 | 0.180 | 5 | 4 | 5 |
| | containing family | | | | | | |
| | B member 1 | | | | | | |
| Q8TAX7 | Mucin-7 | 0.021 | 0.009 | 0.020 | 5 | 5 | 5 |
| P07355-2 | Annexin A2 | 0.041 | 0.018 | 0.066 | 5 | 5 | 5 |
| P23280-2 | Carbonic | 0.302 | 0.148 | 0.294 | 5 | 5 | 5 |
| | anhydrase 6 | | | | | | |
| P35527 | Keratin, type I | 1.464 | 0.803 | 1.146 | 5 | 5 | 5 |
| | cytoskeletal 9 | | | | | | |
| P01011 | Alpha-1- | 0.035 | 0.020 | 0.043 | 5 | 4 | 5 |
| | antichymotrypsin | | | | | | |
| P01700 | Ig lambda | 0.081 | 0.052 | 0.068 | 5 | 5 | 5 |
| | variable 1-47 | | | | | | |

| | Detection count | | | | |
|---|---|---|---|---|---|
| | | Before | After | Ratio | |
| | Healthy | Treatment | Treatment | Healthy/ | GO Biological |
| Uniprot ID | group | group | group | Before | Function |
| P00558 | 15 | 14 | 15 | 4.452 | canonical glycolysis |
| Q6UWP8 | 15 | 13 | 15 | 3.197 | NA |
| Q8TDL5 | 15 | 11 | 15 | 3.021 | antimicrobial humoral response |
| Q8TAX7 | 15 | 14 | 15 | 2.385 | antimicrobial humoral immune |
| | | | | | response |
| P07355-2 | 15 | 12 | 15 | 2.304 | angiogenesis/osteoclast |
| | | | | | development/neutrophil |
| | | | | | degranulation |
| P23280-2 | 15 | 14 | 15 | 2.041 | bicarbonate transport |
| P35527 | 15 | 14 | 15 | 1.822 | keratinization |
| P01011 | 15 | 12 | 15 | 1.758 | acute-phase response/ |
| | | | | | inflammatory response |
| P01700 | 15 | 14 | 15 | 1.554 | immune response |

TABLE 4

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| P30086 | Phosphatidylethanolamine- | 0.204 | 0.049 | 0.149 | 5 | 5 | 5 |
| | binding protein 1 | | | | | | |
| Q02413 | Desmoglein-1 | 0.024 | 0.007 | 0.022 | 5 | 5 | 5 |
| P25311 | Zinc-alpha-2-glycoprotein | 2.057 | 1.025 | 2.141 | 5 | 5 | 5 |

| | Detection count | | | | |
|---|---|---|---|---|---|
| | | Before | After | Ratio | |
| | Healthy | Treatment | Treatment | Healthy/ | GO Biological |
| Uniprot ID | group | group | group | Before | Function |
| P30086 | 15 | 15 | 15 | 4.207 | MAPK cascade |
| Q02413 | 15 | 15 | 15 | 3.583 | cell-cell adhesion/ |
| | | | | | keratinization |
| P25311 | 15 | 15 | 15 | 2.007 | cell adhesion/immune |
| | | | | | response |

Here, the treatment of periodontal disease or the resolution of inflammation were determined as an improvement in all clinical values compared with before treatment. Specifically, compared with before treatment, the periodontal pocket depth and clinical attachment level of all teeth were reduced, and the percentage of bleeding on probing (BOP) among all teeth decreased from 69.71% (means that 69.71% of all tooth surfaces exhibit BOP) to 24.10% after treatment. In addition, the percentage of a region in which a periodontal pocket depth, which is an indicator showing the distribution of sites where severe periodontitis occurred, is 5 mm or more was 38.4% before treatment, but decreased to 7.8% after treatment on average. It was evaluated that periodontal disease was cured or inflammation was resolved through the decrease in indicators showing the severity of periodontitis (see Table 5).

Accordingly, to provide information required for the diagnosis of a periodontal condition, the present invention may provide a method of detecting a marker protein for diagnosing a periodontal condition, which includes:

i) detecting one or more proteins listed in Tables 1A, 1B, 2, 3 and 4 from a subject's sample; and

ii) associating the subject with the diagnosis of a periodontal condition, if the above-listed one or more proteins are increased or decreased in concentration in comparison with a control sample.

In the method of the present invention, compared with a normal control sample, if one or more proteins listed in Table 1A increase in concentration, and/or one or more proteins listed in Tables 1B, 2, 3 and 4 decrease in concentration, the case may be determined as an abnormal periodontal condition.

In addition, compared with an abnormal control sample, if one or more proteins listed in Table 1A decrease in concentration, and/or one or more proteins listed in Table 1B, 2, 3 and 4 increase in concentration, the case may be determined as a normal periodontal condition.

The normal control may be a sample of a subject who has not developed periodontal disease or a subject whose disease symptoms were treated by receiving treatment with a drug or medical procedure, and the abnormal control may be a sample of a subject who has developed periodontal disease.

In one embodiment, the determination may be performed by comparing the result of detecting a protein marker listed in the tables (protein expression level or concentration) with a threshold for each marker determined in a control.

In one embodiment, for each protein marker, a normal or abnormal range of values relative to the threshold may be determined. For example, when the value of a corresponding marker in a subject's sample increases by approximately 30%, 50% or 70% or more, compared with the threshold, it may be diagnosed as an abnormal condition in which periodontal disease is suspected or a normal periodontal condition.

Or/at the same time, the value of a corresponding marker in a subject's sample decreases by approximately 30%, 50% or 70% or more, compared with the threshold, it may be diagnosed as an abnormal condition in which periodontal disease is suspected or a normal periodontal condition.

In one embodiment, after a ratio of a detection value of a corresponding marker in a subject's sample and a detection value of a corresponding marker of a control is calculated, when the ratio is 2.0 or more, it may be diagnosed as an abnormal condition in which periodontal disease is suspected or a normal periodontal condition.

In addition, the determination may further increase the accuracy of diagnosis when a combination of several protein markers listed in Tables 1A, 1B, 2, 3 and 4 is determined.

Meanwhile, the method of the present invention may be used along with conventional clinical information which has been used in diagnosis of periodontal disease to further increase the accuracy of diagnosis. The clinical information includes a periodontal pocket depth, bone loss on a radiograph, and bleeding on probing.

The proteins listed in Table 1A are proteins which were repeatedly detected three times in all samples of 5 persons with periodontal disease, but not detected in samples from the individual having healthy gums or in which inflammation was resolved after the treatment of periodontitis in all experiments, and whose ratios are two-fold or more different when detected.

The amino acid sequences of the listed proteins are shown by corresponding Uniprot IDs from the known gene database, UniProt (www.uniprot.org). Specifically, Hemoglobin subunit delta, Histone H3.1, Neutrophil collagenase, Myosin-9, WD repeat-containing protein 1, Cathepsin G, Serpin B10, Vimentin and Protein S100-P may have Uniprot ID registration numbers in the order of P02042, P68431, P22894, P35579, O75083, P08311, P48595, B0YJC4 and P25815, and have amino acid sequences in the order of SEQ ID NOs: 1 to 9 in the sequence listing, respectively.

The proteins listed in Table 1B are proteins which were repeatedly detected three times in all samples of 5 persons with periodontal disease, but not detected in a sample from an individual having healthy gums or in which inflammation was resolved after periodontal disease treatment in all experiments, and whose ratios in the healthy group are two-fold or more different from those of the periodontal disease group when detected.

The amino acid sequences of the listed proteins are shown by corresponding Uniprot IDs in the known gene database, UniProt (www.uniprot.org). Specifically, Heme-binding protein 2, Alpha-actinin-4, and Protein disulfide-isomerase may have Uniprot ID registration numbers in the order of Q9Y5Z4, O43707 and P07237, and have amino acid sequences in the order of SEQ ID NOs: 10 to 12 in the sequence listing, respectively.

The proteins listed in Table 2 are 19 proteins which were detected repeatedly three times in all samples of 5 persons with healthy periodontal tissue, but not detected in groups before and after periodontal treatment in all experiments, and whose ratios are two-fold or more different between the healthy group and the periodontal disease groups.

The amino acid sequences of the listed proteins are shown by corresponding Uniprot IDs from the known gene database, UniProt (www.uniprot.org). Specifically, Aldo-keto reductase family 1 member B10, Desmocollin-2, BPI fold-containing family A member 1, Delta and Notch-like epidermal growth factor-related receptor, Phospholipid transfer protein, Ganglioside GM2 activator, Ig heavy variable 3-49, Isoform 2 of Interleukin-1 receptor antagonist protein, BPI fold-containing family A member 2, Carcinoembryonic antigen-related cell adhesion molecule 6, Isoform 2 of Clusterin, KRT6A Keratin, type II cytoskeletal 6A, Leucine-rich alpha-2-glycoprotein, Lactoperoxidase, Ig heavy constant alpha 2, Ig lambda constant 2, Deleted in malignant brain tumors 1 protein, Ig heavy constant mu and Glyceraldehyde-3-phosphate dehydrogenase may have Uniprot ID registration numbers in the order of O60218, Q02487, Q9NP55, Q8NFT8, P55058, P17900, A0A0A0MS15, P18510-2, Q96DR5, P40199, P10909-2, P02538, P02750, P22079, A0A0G2JMB2, P0DOY2, Q9UGM3, P01871 and P04406, and have amino acid sequences in the order of SEQ ID NOs: 13 to 31 in the sequence listing, respectively.

The proteins listed in Table 3 are 9 proteins which were detected repeatedly three times in all samples of 5 persons with healthy periodontal tissue and samples of 5 persons after periodontal treatment, but not detected in the periodontitis group in all experiments, and whose ratios are 1.5-fold or more different between the healthy group and the periodontal disease group when detected.

The amino acid sequences of the listed proteins are shown as corresponding Uniprot IDs in the known gene database, UniProt (www.uniprot.org). Specifically, Phosphoglycerate kinase 1, Suprabasin, BPI fold-containing family B member 1, Mucin-7, Annexin A2, Carbonic anhydrase 6, Keratin, type I cytoskeletal 9, Alpha-1-antichymotrypsin and Ig lambda variable 1-47 may have Uniprot ID registration numbers in the order of P00558, Q6UWP8, Q8TDL5, Q8TAX7, P07355-2, P23280-2, P35527, P01011 and P01700, and have amino acid sequences in the order of SEQ ID NOs: 32 to 40 in the sequence listing, respectively.

In Table 4, among 60 proteins repeatedly detected three times in all of samples of 5 persons with normal gums, 5 persons whose inflammation was resolved after periodontal disease treatment and 5 persons with periodontal disease, three proteins having an average mol % in the healthy group that is at least twice that in the periodontal disease group are listed.

The amino acid sequences of the listed proteins are shown by corresponding Uniprot IDs in the known gene database, UniProt (www.uniprot.org). Specifically, Phosphatidylethanolamine-binding protein 1, Desmoglein-1 and Zinc-alpha-2-glycoprotein may have Uniprot ID registration numbers in the order of P30086, Q02413 and P25311, and have amino acid sequences in the order of SEQ ID NOs: 41 to 43 in the sequence listing, respectively.

In one embodiment, a process of detecting a marker protein from a subject's sample may be performed using a method generally known in the art.

For example, the detection of the marker protein may be performed by a method of detecting an antigen-antibody complex formed by an antibody against a marker protein or a fragment thereof through western blotting, enzyme-linked immunosorbent assay (ELISA), immunoprecipitation assay, complement fixation assay, radio immunoassay (RIA) or fluorescence activated cell sorting (FACS), which are known in the art.

Specifically, a sandwich-type immunoassay such as ELISA may be used.

The immunoassay method is described in, for example, Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, N.J., 1984. By analyzing the intensity of the final signal by the above-described immunoassay process, that is, by performing a signal comparison with a control specimen, the diagnosis of a symptom, disease or condition may be associated with.

The detection reagent used in the above-described method may include, for example, a monoclonal antibody, a polyclonal antibody, a substrate, an aptamer, an avimer, a peptidomimetic, a receptor, a ligand or a cofactor.

In one embodiment, the detection reagent is an antibody specifically binding to a marker protein according to the present invention or a fragment thereof, and thus is able to quantitatively or qualitatively analyze a protein in a sample.

In addition, according to another aspect of the present invention, a composition for diagnosing a periodontal condition, which includes an antibody specifically binding to a protein, listed in Tables 1A, 1B, 2, 3 or 4, present in saliva, or a fragment thereof, may be provided. The composition may be used to diagnose or assist in diagnosing a periodontal condition.

The antibody may be used to measure the presence or absence or expression level of a protein in a sample, and as a monoclonal antibody, a polyclonal antibody or a recombinant antibody, is specific for the marker protein or a fragment thereof. A monoclonal antibody may be prepared using a hybridoma method (Kohler and Milstein (1976), European Journal of Immunology 6:511-519) or a phage antibody library technique (Clarkson et al, Nature, 352:624-628, 1991; Marks et al, J. Mol. Biol., 222:58, 1-597, 1991), which is widely known in the art. A polyclonal antibody may be prepared by a method known in the art, which includes injecting a protein antigen into an animal and obtaining serum containing an antibody from the animal. The polyclonal antibody is able to be prepared from any animal including a dog, a goat, sheep, a rabbit, a monkey, a horse, a pig and a cow. In addition, the antibody includes a chimeric antibody, a humanized antibody, and a human antibody. Further, the antibody may include a full antibody with two full-length light chains and two full-length heavy chains or a functional fragment thereof. The functional fragment of the antibody refers to a fragment possessing an antigen-binding function and includes Fab, F(ab′), F(ab′)2 and Fv.

In addition, the present invention may provide a kit for diagnosing a periodontal condition, which includes an agent for measuring the presence or absence or expression level of one or more proteins selected from Tables 1A, 1B, 2, 3 and 4, as a protein whose expression level is relatively increased in a subject's sample.

The kit may include one or more compositions, solutions or devices suitable for analysis of a protein level, in addition to an agent for measuring an expression level of the protein marker in a subject's sample. For example, the kit may include an antibody for recognizing a marker, a substrate for immunological detection of the antibody, a buffer solution, a secondary antibody labeled with a detection label, and a chromogenic substrate.

For example, the kit may include a component necessary for performing an ELISA method such as an ELISA kit, a sandwich ELISA, etc. That is, the ELISA kit may include an antibody specific for the protein marker, and include reagents capable of detecting a bound antibody, for example, a labeled secondary antibody, a chromophore, an enzyme and the substrate thereof, or other materials that are able to bind to an antibody.

The diagnostic kit may be a kit for western blotting, immunoprecipitation assay, complement fixation assay, flow cytometry or a microarray.

In addition, the kit of the present invention may include one or more additional components required for analysis, and may further include, for example, a buffer solution required for detection, a reagent required for sample preparation, a tool for sampling, negative and/or positive controls, and instructions on the use of a biomarker.

The kit for periodontal diagnosis using a protein marker of the present invention may help in easily diagnosing whether the gums are healthy in other areas of internal medicine requiring discrimination to determine whether periodontitis serves as a cause of a systemic disease such as a cardiovascular disease, premature birth, diabetes, cerebrovascular disease or pneumonia, which is known to be closely related to periodontitis.

It is important to develop a diagnostic kit which can show whether periodontal disease is currently progressing or tissue destruction has stopped after treatment, and it is difficult for current diagnostic methods to show a current condition of the progression of tissue and alveolar bone destruction by periodontitis, and the current methods have a limitation in that a therapeutic effect and the termination of tissue destruction after treatment cannot be shown.

A periodontal diagnosis method using the detection of a salivary protein marker of the present invention may clearly show whether actual inflammation occurs in gum tissue by detecting proteins in saliva involved in an inflammatory response, which originate from gingival tissue.

For example, the presence of the proteins in saliva at a relatively low concentration may represent a state in which there is no or reduced periodontal inflammatory response. Among the proteins listed in Table 1, Hemoglobin subunit delta and Histone H3.1 may be markers which are able to show the leakage of blood through vasodilation due to an inflammatory response in gingival tissue. Serpin B10, Protein S100-P, etc. may show the activation of neutrophils in gingival tissue. Neutrophil collagenase is an indicator showing a protease in gingival tissue, and an increase of the protein in concentration may show the possible destruction of gingival tissue. As such, the increase of a specific protein in saliva may act as an indicator directly showing an inflammatory response in gingival tissue.

The proteins in Table 1A, 1B, 2, 3 and 4 of the present invention are proteins having a great difference in expression (including its presence or absence) in saliva between normal and abnormal gums, and show similar results in three repeated experiments with all 5 persons in a group. Therefore, they may be used as a generally applicable marker for diagnosing a periodontal condition, or may be effectively used as an auxiliary indicator for diagnosing a periodontal condition.

### EXAMPLES

### 1. Experimental Procedures and Materials

### 1-1: Research Participants and Saliva Sampling

The study was conducted by obtaining saliva samples from 5 subjects with healthy gums and 5 persons diagnosed with periodontitis and performing analysis thereon. For this study, prior consent was obtained from all subjects to obtain saliva samples at Ajou University Dental Hospital and Seoul St. Mary's Hospital, and the study was approved by the Institutional Review Board for Human Subjects of Ajou University Dental Hospital (AJIRB-BMR-SMP-16) and Seoul St. Mary's Hospital (KC16TIMI0755).

No subjects with orthodontic appliances, other systemic histories that can affect the progression of periodontitis or a smoking habit were included in this study, and subjects taking antibiotics and anti-inflammatory drugs for 3 months prior to testing and sampling were excluded.

Those having an average periodontal pocket depth of less than 3 mm and a bleeding on probing area of less than 20% were classified as subjects with healthy gums. Those having 25 or more teeth in the mouth and 8 or more teeth with sites having a probing pocket depth (PD) of 5 mm were classified as periodontitis patients. All subjects were instructed to avoid food intake, rinsing (gargling) and brushing 1 hour before saliva sampling. From each subject, approximately 3 mL of a non-stimulated whole saliva sample was obtained and centrifuged at 13,200 rpm for 5 minutes to remove bacteria and impurities in saliva, and a supernatant was obtained and stored at −80° C. until analysis. Patients with periodontitis received a total of four non-surgical periodontal treatments, and saliva sampling and measurements of clinical parameters were repeated 3 months after treatment.

### 1-2: Evaluation of Clinical Indicators

Saliva donors included 5 subjects (5 females) having healthy gums with an average age of 37 years (25 to 47 years) and 5 subjects (3 males and 2 females) with periodontitis with an average age of 52 years (44 to 57 years). They were divided into a total of three groups (a periodontally healthy (normal) group, a group before treatment of periodontitis, and a group after treatment of periodontitis) for analysis. The clinical indicators for these three groups are summarized in Table 5.

TABLE 5

| | | Periodontitis | |
|---|---|---|---|
| | | Before | After |
| Clinical indicator | Normal | treatment | treatment |
| The sum of whole | 19.6 (6/35) | 38.4 (9/76) | 11.6 (2/30) |
| mouth PI | | | |
| PD (mm) | 2.19 ± 0.08 | 3.72 ± 0.14 | 2.59 ± 0.23 |
| CAL (mm) | 2.29 ± 0.12 | 4.03 ± 0.21 | 3.14 ± 0.40 |
| Count of sites | 0 | 38.40 ± 4.23 | 7.80 ± 5.58 |
| with PD ≥ 5 mm | | | |
| % BOP | 10.00 ± 2.09 | 69.71 ± 8.85 | 24.10 ± 6.46 |

In Table 5, the sum of whole mouth PI refers to the sum of plaque indices (0=No plaque, 1=Island-type plaque without connection, 2=Line-type plaque along the gingival margin, 3=Plaque formed on ⅓ of the cervical margin, 4=Plaque formed on ⅔ of the crown, and 5=Plaque covering almost all of the crown; indicating that the larger the number, the more plaque that causes periodontitis) on the outside and inside of all teeth in the mouth. PI is 19.6 in the normal group, 38.4 in the patient group before treatment of periodontitis and 11.6 in the group after treatment of periodontitis, indicating that the PIs of the normal group and the group after treatment of periodontitis are lower than that of the group before treatment of periodontitis.

The probing pocket depth (PD) is an average for all teeth of lengths measured by inserting a periodontal probe between a tooth and a gum at a total of six areas including 3 areas each outside and inside one tooth, and the higher the value, the more severe the periodontitis. The average PD for all teeth is 2.19 in a normal group, whereas the average PD is 3.72 before periodontitis treatment, indicating a higher level of average probing depth was shown in a periodontitis group, compared with the normal group. It can be seen that, after the treatment of periodontitis, the average PD was 2.59, which was lower than that before the treatment of periodontitis.

The clinical attachment level (CAL) is a value obtained by adding a gingival recession from the cementoenamel junction of a tooth to the gingival margin to probing depths measured using a periodontal probe, for a total of six areas, including 3 areas each outside and inside of one tooth, and the values in the table are average CALs for the all teeth in the mouth. Like the probing depth, the larger the value, the more severe the degree of periodontitis. In the normal group, the average CAL for all teeth is 2.29, whereas before the treatment of periodontitis, the average CAL is 4.03, indicating a higher level of average CAL in the periodontitis group than that of the normal group. It can be seen that after the periodontitis treatment, the average CAL is 3.14, which is lower than that before the treatment of periodontitis.

A higher PD value indicates an increase in tissue destruction caused by periodontitis, and 5-mm or more periodontal pockets may indicate that bone destruction occurred due to severe periodontitis. In the normal group, there was no place having a periodontal pocket depth of 5 mm or more among all teeth, whereas before the treatment of periodontitis, the average number of sites where the above-mentioned periodontal pockets appear is 38.4, indicating that there is more severe bone destruction before the treatment of periodontitis, compared with a normal person. On the other hand, it was confirmed that, after the treatment of periodontitis, the average number of sites where 5-mm or more periodontal pockets appear is 7.8, which was significantly lower than that before the treatment of periodontitis.

% BOP is an average value of the number of sites where bleeding occurs during the measurement of a periodontal pocket at one tooth for all teeth, expressed as a percentage, and the higher the value, the more severe the gingival inflammation in the mouth. The average % BOP is 10.00 in the healthy group, but 69.71 in the group before the treatment of periodontitis, indicating that there was a lot of periodontal inflammation before the treatment of periodontitis. The value after the treatment of periodontitis was 24.10, which is significantly lower than that before treatment and indicates that gingival inflammation was reduced by periodontitis treatment.

As described above, 3 months after non-surgical treatment, the improvement in all clinical indicators of periodontitis patients was observed, and it was evaluated that periodontal inflammation was resolved by the non-surgical treatment.

### 1-3: Protein Analysis Method (LS-MS/MS)

Prepared saliva samples were dissolved again in a sodium dodecyl sulphate-polyacrylamide gel electrophoresis (SDS-PAGE) buffer (1 M TRIS-HCl pH 6.8, 10% SDS, 1% bromophenol blue, glycerol and β-mercaptoethanol), and heated for 10 minutes. The resulting products were subjected to electrophoresis with 12% SDS-PAGE, the gel was stained with Coomassie Brilliant Blue R-250, and proteins were then separated according to molecular weight (FIG. 2). After tryptic in-gel digestion, the resulting products were isolated with an extraction solution consisting of 50 mM ammonium bicarbonate, 50% acetonitrile and 5% trifluoroacetic acid (TFA) and then dried. For the subsequent LC-MS/MS analysis, the resulting product was dissolved in 0.5% TFA.

A tryptic peptide sample was separated using an Ultimate 3000 UPLC system (Dionex, Sunnyvale, Calif., USA) connected with a nano-electrospray ionization source (Dionex)-linked Q Exactive Plus mass spectrometer (Thermo Scientifc, Waltham, Mass., USA). The peptide separated through a column was transferred to a 15 cm×75 μm i.d. Acclaim PepMap RSLC C18 reverse-phase analytical column (Thermo Scientifc) at a rate of 300 nanoliters/min, and separated using gradient elution of 0 to 65% acetonitrile in 0.1% formic acid for 3 hours.

All MS and MS/MS spectra were acquired in a data-dependent top-ten mode in an auto conversion method. A survey full-scan MS spectrum (m/z 150-2,000) was acquired with 70,000 (m/z 200) resolution. The MS/MS spectra were searched against a UniProt human database using MASCOT v2.4 (Matrix Science, Inc., Boston, Mass., USA). As data base search conditions, 1) carbamidomethylation of cysteine, 2) oxidation of methionine, 3) two instances of failed trypsin decomposition, and 4) a mass difference between a precursor ion and a product ion of 10 ppm or less were selected. Each sample was repeatedly analyzed 3 times.

### 2. Selection of Protein Marker

### 2-1: Result of Total Protein Detection

Through three repeated experiments by the above-described protein analysis method, the number of proteins detected in each of 5 samples in a periodontally healthy group and 5 samples in the groups before the treatment of periodontitis and 5 samples in the group after the treatment of periodontitis was a total of 168.

Referring to FIG. 1, a total of 101 proteins in a periodontally healthy group, a total of 110 proteins in a periodontitis patient group before treatment, and a total of 109 proteins in a periodontitis patient group after treatment were detected. The number of proteins detected in common in all three groups was 60. The number of proteins found only in the periodontitis patient group before treatment was 26 and in the healthy group, was 24, and the number of proteins found in common in both of the healthy group and the post-treatment group was 11. Among the 60 proteins found in all of the periodontally healthy group, the patient group before the treatment of periodontitis and the group after the treatment of periodontitis, 57 proteins were excluded from biomarker candidates because of insignificant differences in concentration between groups.

Likewise, 66 proteins detected in common in the periodontally healthy group and the patient group before the treatment of periodontitis, and 78 proteins found before the treatment and detected even after the treatment were excluded from biomarker candidates indicating periodontal disease or healthy gums.

### 2-2: Selection of Protein Marker of Periodontal Disease

Among 26 proteins in Table A, which were repeatedly detected three times in all five samples in a group having inflamed periodontal tissue using the proteomics analysis of the present invention, but not detected in the healthy group or the post-treatment group, proteins having a 2-fold or more difference from the average protein concentration in the healthy group or the post-treatment group are shown in Tables 1A and 1B. The concentrations of Hemoglobin subunit delta, Histone H3.1, Neutrophil collagenase, Myosin-9, WD repeat-containing protein 1, Cathepsin G, Serpin B10, Vimentin and Protein S100-P in Table 1A ranged from 1.906 to 0.069 mol % before the treatment, which is 956.388 to 2.159-fold higher than that of the healthy group. Heme-binding protein 2, Alpha-actinin-4 and Protein disulfide-isomerase in Table 1B were detected three times in all 5 samples in the periodontitis group, but not detected in the healthy group or the post-treatment group in all of the experiments, and detected at 2-fold or more in the healthy group, when comparing the average.

In Table A, the protein mol % of a corresponding protein per group, the number of persons detected per group, and the number of detections in a total of 15 repeated experiments are recorded, and listed in order of ratios of the patient group (before treatment)/healthy group.

The 26 proteins in Table A were subjected to three repeated analyses of salivary proteins from 5 patients with periodontal disease and detected in all three analyses, and recorded with an average of concentrations measured three times. The fact that a protein was not detected in the periodontally healthy group and the group after the treatment of periodontitis means that, when a subject's saliva was analyzed three times, the corresponding protein was detected less than 2 times or not detected at all. The amino acid sequences of corresponding proteins may be confirmed by corresponding Uniprot IDs from UniProt (www.uniprot.org), and are represented by SEQ ID NOs: 1 to 9 of the accompanying sequence listing. The GO biological function shown in Table A indicates the function of a corresponding protein. When indicated as NA, it means that the function of a corresponding protein is not known.

TABLE A

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| P02042 | Hemoglobin | 0.002 | 1.906 | 0.719 | 1 | 5 | 2 |
| | subunit delta | | | | | | |
| P68431 | Histone H3.1 | 0.007 | 0.130 | 0.082 | 1 | 5 | 4 |
| P22894 | Neutrophil | 0.008 | 0.024 | 0.019 | 4 | 5 | 5 |
| | collagenase | | | | | | |
| P35579 | Myosin-9 | 0.004 | 0.012 | 0.017 | 4 | 5 | 5 |
| O75083 | WD repeat- | 0.007 | 0.018 | 0.012 | 5 | 5 | 4 |
| | containing | | | | | | |
| | protein 1 | | | | | | |
| P08311 | Cathepsin G | 0.022 | 0.059 | 0.063 | 2 | 5 | 4 |
| P48595 | Serpin B10 | 0.008 | 0.020 | 0.018 | 4 | 5 | 4 |
| B0YJC4 | Vimentin | 0.011 | 0.027 | 0.018 | 2 | 5 | 4 |
| P25815 | Protein | 0.032 | 0.069 | 0.046 | 5 | 5 | 5 |
| | S100-P | | | | | | |
| Q6UX06 | Olfactomedin-4 | 0.018 | 0.031 | 0.036 | 4 | 5 | 4 |
| P46940 | Ras GTPase- | 0.004 | 0.007 | 0.006 | 4 | 5 | 5 |
| | activating- | | | | | | |
| | like protein | | | | | | |
| | IQGAP1 | | | | | | |
| U3KPS2 | Myeloblastin | 0.036 | 0.055 | 0.054 | 5 | 5 | 5 |
| Q15084-5 | Isoform 5 of | 0.005 | 0.007 | 0.006 | 5 | 5 | 4 |
| | Protein | | | | | | |
| | disulfide- | | | | | | |
| | isomerase | | | | | | |
| | A6 | | | | | | |
| P00738 | Haptoglobin | 0.117 | 0.152 | 0.098 | 4 | 5 | 5 |
| A0A286YFJ8 | Ig heavy | 0.090 | 0.117 | 0.094 | 5 | 5 | 4 |
| | constant | | | | | | |
| | gamma 4 | | | | | | |
| P19652 | Alpha-1-acid | 0.033 | 0.039 | 0.043 | 5 | 5 | 4 |
| | glycoprotein | | | | | | |
| | 2 | | | | | | |
| P52907 | F-actin- | 0.027 | 0.031 | 0.041 | 5 | 5 | 5 |
| | capping | | | | | | |
| | protein | | | | | | |
| | subunit | | | | | | |
| | alpha-1 | | | | | | |
| O00764 | Pyridoxal | 0.022 | 0.024 | 0.032 | 4 | 5 | 5 |
| | kinase | | | | | | |
| P09960 | Leukotriene | 0.031 | 0.026 | 0.045 | 5 | 5 | 5 |
| | A-4 | | | | | | |
| | hydrolase | | | | | | |
| P11142 | Heat shock | 0.065 | 0.052 | 0.074 | 4 | 5 | 4 |
| | cognate 71 | | | | | | |
| | kDa protein | | | | | | |
| P31946 | 14-3-3 | 0.173 | 0.121 | 0.127 | 4 | 5 | 5 |
| | protein | | | | | | |
| | beta/alpha | | | | | | |
| P09972 | Fructose- | 0.040 | 0.026 | 0.029 | 5 | 5 | 4 |
| | bisphosphate | | | | | | |
| | aldolase C | | | | | | |
| P61769 | Beta-2- | 0.137 | 0.085 | 0.136 | 5 | 5 | 5 |
| | microglobulin | | | | | | |
| Q9Y5Z4 | Heme- | 0.026 | 0.013 | 0.012 | 5 | 5 | 5 |
| | binding | | | | | | |
| | protein 2 | | | | | | |
| O43707 | Alpha- | 0.102 | 0.047 | 0.064 | 5 | 5 | 5 |
| | actinin-4 | | | | | | |
| P07237 | Protein | 0.080 | 0.015 | 0.060 | 5 | 5 | 5 |
| | disulfide- | | | | | | |
| | isomerase | | | | | | |

| | Detection count | | | | |
|---|---|---|---|---|---|
| | | Before | After | Ratio | |
| | Healthy | Treatment | Treatment | Before/ | GO Biological |
| Uniprot ID | group | group | group | Healthy | Function |
| P02042 | 1 | 15 | 5 | 956.388 | blood coagulation |
| P68431 | 2 | 15 | 11 | 18.571 | blood coagulation |
| P22894 | 10 | 15 | 12 | 2.962 | collagen catabolic process/ |
| | | | | | neutrophil degranulation |
| P35579 | 11 | 15 | 14 | 2.845 | actin cytoskeleton |
| | | | | | reorganization/leukocyte |
| | | | | | migration |
| O75083 | 13 | 15 | 12 | 2.701 | actin filament |
| | | | | | depolymerization/neutrophil |
| | | | | | mediated immunity |
| P08311 | 6 | 15 | 12 | 2.695 | angiotensin |
| | | | | | maturation/antibacterial |
| | | | | | humoral response |
| P48595 | 9 | 15 | 11 | 2.581 | negative regulation of |
| | | | | | endopeptidase activity/ |
| | | | | | neutrophil degranulation |
| B0YJC4 | 5 | 15 | 7 | 2.408 | NA |
| P25815 | 13 | 15 | 14 | 2.159 | endothelial cell |
| | | | | | migration/neutrophil |
| | | | | | degranulation |
| Q6UX06 | 11 | 15 | 12 | 1.722 | cell adhesion/neutrophil |
| | | | | | degranulation |
| P46940 | 10 | 15 | 14 | 1.546 | cell migration/neutrophil |
| | | | | | degranulation |
| U3KPS2 | 12 | 15 | 13 | 1.502 | NA |
| Q15084-5 | 9 | 15 | 9 | 1.382 | cell redox homeostasis |
| P00738 | 12 | 15 | 11 | 1.304 | acute inflammatory |
| | | | | | response |
| A0A286YFJ8 | 12 | 15 | 10 | 1.295 | NA |
| P19652 | 11 | 15 | 11 | 1.175 | acute-phase response |
| P52907 | 12 | 15 | 13 | 1.172 | actin cytoskeleton |
| | | | | | organization/innate immune |
| | | | | | response |
| O00764 | 10 | 15 | 14 | 1.134 | cell population proliferation/ |
| | | | | | neutrophil degranulation |
| P09960 | 13 | 15 | 11 | 0.832 | cellular lipid metabolic |
| | | | | | process/neutrophil |
| | | | | | degranulation |
| P11142 | 8 | 15 | 11 | 0.804 | ATP metabolic process/ |
| | | | | | neutrophil degranulation |
| P31946 | 11 | 15 | 14 | 0.699 | cytoplasmic sequestering of |
| | | | | | protein |
| P09972 | 13 | 15 | 12 | 0.632 | canonical glycolysis/ |
| | | | | | neutrophil degranulation |
| P61769 | 14 | 15 | 13 | 0.623 | antibacterial humoral |
| | | | | | response |
| Q9Y5Z4 | 14 | 15 | 13 | 0.488 | negative regulation of |
| | | | | | mitochondrial membrane |
| | | | | | potential/ neutrophil |
| | | | | | degranulation |
| O43707 | 14 | 15 | 14 | 0.460 | actin filament bundle |
| | | | | | assembly/platelet |
| | | | | | degranulation |
| P07237 | 14 | 15 | 13 | 0.191 | cell redox |
| | | | | | homeostasis/cellular |
| | | | | | response to IL7, 12, 23 |

The detailed detection count and amount of each protein are shown in Table A-1.

TABLE A-1

| | | | | | | Protein mol %(Individual Average) | | | | |
|---|---|---|---|---|---|---|---|---|---|---|
| | Protein | | | | | Healthy group | | | | |
| Uniprot ID | description | | | | | 1 | 2 | 3 | 4 | 5 |
| P02042 | Hemoglobin subunit delta | | | | | 0.000 | 0.010 | 0.000 | 0.000 | 0.000 |
| P52907 | F-actin-capping protein subunit | | | | | 0.036 | 0.032 | 0.048 | 0.007 | 0.009 |
| | alpha-1 | | | | | | | | | |
| P00738 | Haptoglobin | | | | | 0.136 | 0.042 | 0.231 | 0.000 | 0.175 |
| B0YJC4 | Vimentin | | | | | 0.000 | 0.009 | 0.048 | 0.000 | 0.000 |
| P68431 | Histone H3.1 | | | | | 0.000 | 0.000 | 0.035 | 0.000 | 0.000 |
| P09960 | Leukotriene A04 hydrolase | | | | | 0.056 | 0.021 | 0.067 | 0.005 | 0.007 |
| P31946 | 14-3-3 protein beta/alpha | | | | | 0.389 | 0.153 | 0.288 | 0.000 | 0.036 |
| P09972 | Fructose-bisphosphate aldolase C | | | | | 0.094 | 0.023 | 0.069 | 0.007 | 0.009 |
| A0A286YFJ8 | Ig heavy constant gamma 4 | | | | | 0.112 | 0.112 | 0.059 | 0.132 | 0.036 |
| O00764 | Pyridoxal kinase | | | | | 0.029 | 0.018 | 0.059 | 0.002 | 0.000 |
| P61769 | Beta-2-microglobulin | | | | | 0.105 | 0.122 | 0.101 | 0.282 | 0.075 |
| P22894 | Neutrophil collagenase | | | | | 0.003 | 0.005 | 0.012 | 0.000 | 0.020 |
| P25815 | Protein S100-P | | | | | 0.022 | 0.037 | 0.052 | 0.023 | 0.026 |
| P48595 | Serpin B10 | | | | | 0.009 | 0.002 | 0.025 | 0.000 | 0.003 |
| P08311 | Cathepsin G | | | | | 0.026 | 0.000 | 0.083 | 0.000 | 0.000 |
| O75083 | WD repeat-containing protein 1 | | | | | 0.008 | 0.004 | 0.017 | 0.001 | 0.003 |
| U3KPS2 | Myeloblastin | | | | | 0.028 | 0.034 | 0.040 | 0.029 | 0.050 |
| P07237 | Protein disulfide-isomerase | | | | | 0.199 | 0.023 | 0.166 | 0.007 | 0.006 |
| P11142 | Heat shock cog0te 71 kDa protein | | | | | 0.157 | 0.008 | 0.150 | 0.010 | 0.000 |
| Q9Y5Z4 | Heme-binding protein 2 | | | | | 0.045 | 0.015 | 0.048 | 0.008 | 0.015 |
| O43707 | Alpha-actinin-4 | | | | | 0.171 | 0.045 | 0.279 | 0.010 | 0.006 |
| P35579 | Myosin-9 | | | | | 0.001 | 0.012 | 0.008 | 0.000 | 0.000 |
| P19652 | Alpha-1-acid glycoprotein 2 | | | | | 0.054 | 0.013 | 0.024 | 0.030 | 0.044 |
| Q15084-5 | Isoform 5 of Protein disulfide- | | | | | 0.010 | 0.002 | 0.011 | 0.001 | 0.001 |
| | isomerase A6 | | | | | | | | | |
| Q6UX06 | Olfactomedin-4 | | | | | 0.000 | 0.036 | 0.030 | 0.010 | 0.015 |
| P46940 | Ras GTPase0activating-like protein | | | | | 0.005 | 0.004 | 0.013 | 0.000 | 0.000 |
| | IQGAP1 | | | | | | | | | |
| | Protein mol %(Individual Average) | | | | | | | | | |
| | Before Treatment group | | | | | After Treatment group | | | | |
| Uniprot ID | 1 | 2 | 3 | 4 | 5 | 1 | 2 | 3 | 4 | 5 |
| P02042 | 1.321 | 3.170 | 1.848 | 2.482 | 0.712 | 0.000 | 0.000 | 0.000 | 3.341 | 0.254 |
| P52907 | 0.056 | 0.026 | 0.044 | 0.021 | 0.009 | 0.074 | 0.048 | 0.011 | 0.044 | 0.031 |
| P00738 | 0.087 | 0.138 | 0.188 | 0.108 | 0.240 | 0.100 | 0.157 | 0.012 | 0.052 | 0.170 |
| B0YJC4 | 0.046 | 0.010 | 0.034 | 0.027 | 0.020 | 0.048 | 0.030 | 0.002 | 0.012 | 0.000 |
| P68431 | 0.196 | 0.132 | 0.074 | 0.158 | 0.093 | 0.239 | 0.063 | 0.013 | 0.093 | 0.000 |
| P09960 | 0.046 | 0.034 | 0.025 | 0.011 | 0.013 | 0.125 | 0.052 | 0.018 | 0.004 | 0.027 |
| P31946 | 0.167 | 0.068 | 0.096 | 0.102 | 0.173 | 0.240 | 0.184 | 0.091 | 0.036 | 0.082 |
| P09972 | 0.057 | 0.011 | 0.024 | 0.014 | 0.022 | 0.058 | 0.045 | 0.000 | 0.019 | 0.025 |
| A0A286YFJ8 | 0.159 | 0.071 | 0.097 | 0.138 | 0.120 | 0.168 | 0.102 | 0.000 | 0.167 | 0.032 |
| O00764 | 0.025 | 0.014 | 0.018 | 0.028 | 0.038 | 0.054 | 0.022 | 0.018 | 0.057 | 0.011 |
| P61769 | 0.097 | 0.037 | 0.107 | 0.061 | 0.126 | 0.069 | 0.181 | 0.162 | 0.021 | 0.247 |
| P22894 | 0.032 | 0.010 | 0.031 | 0.019 | 0.028 | 0.032 | 0.008 | 0.004 | 0.039 | 0.015 |
| P25815 | 0.058 | 0.048 | 0.057 | 0.099 | 0.082 | 0.056 | 0.038 | 0.036 | 0.067 | 0.033 |
| P48595 | 0.038 | 0.010 | 0.008 | 0.017 | 0.027 | 0.034 | 0.010 | 0.000 | 0.021 | 0.023 |
| P08311 | 0.094 | 0.068 | 0.041 | 0.061 | 0.030 | 0.106 | 0.057 | 0.051 | 0.101 | 0.000 |
| O75083 | 0.022 | 0.009 | 0.010 | 0.026 | 0.022 | 0.025 | 0.011 | 0.000 | 0.020 | 0.005 |
| U3KPS2 | 0.065 | 0.033 | 0.058 | 0.047 | 0.070 | 0.064 | 0.056 | 0.013 | 0.093 | 0.046 |
| P07237 | 0.024 | 0.009 | 0.009 | 0.015 | 0.019 | 0.213 | 0.075 | 0.002 | 0.005 | 0.004 |
| P11142 | 0.117 | 0.017 | 0.040 | 0.042 | 0.045 | 0.166 | 0.085 | 0.029 | 0.089 | 0.000 |
| Q9Y5Z4 | 0.011 | 0.005 | 0.011 | 0.014 | 0.023 | 0.015 | 0.019 | 0.009 | 0.016 | 0.003 |
| O43707 | 0.081 | 0.030 | 0.027 | 0.055 | 0.043 | 0.205 | 0.038 | 0.003 | 0.053 | 0.018 |
| P35579 | 0.007 | 0.016 | 0.012 | 0.015 | 0.010 | 0.034 | 0.008 | 0.003 | 0.017 | 0.022 |
| P19652 | 0.014 | 0.037 | 0.074 | 0.031 | 0.037 | 0.016 | 0.063 | 0.000 | 0.068 | 0.068 |
| Q15084-5 | 0.013 | 0.002 | 0.004 | 0.006 | 0.009 | 0.013 | 0.007 | 0.001 | 0.008 | 0.000 |
| Q6UX06 | 0.055 | 0.016 | 0.015 | 0.024 | 0.047 | 0.060 | 0.029 | 0.000 | 0.072 | 0.019 |
| P46940 | 0.013 | 0.004 | 0.005 | 0.006 | 0.005 | 0.014 | 0.003 | 0.002 | 0.008 | 0.002 |

### 2-3: Selection of Marker Protein of Healthy Gums

First, among the 26 proteins shown in Table A detected in all of the three repeated analyses for 5 samples of the periodontitis group, when comparing actual detected mol % with that of the healthy group, three proteins detected at 2-fold or higher in the periodontally healthy group were selected and shown in Table 1B. The amino acid sequences of the corresponding proteins may be confirmed by corresponding Uniprot IDs from UniProt (www.uniprot.org), and represented by SEQ ID NOs: 10 to 12 of the accompanying sequence listing. The GO biological function in Table 1B indicates the function of a corresponding protein. When indicated as NA, it means that the function of a corresponding protein is not known.

Subsequently, among the 24 proteins shown in Table B, which were detected in 5 samples of the periodontally healthy group in three repeated experiments, but not detected in the saliva samples in the pre- and post-treatment groups in all experiments, 19 proteins in the healthy group having a mol % 2-fold or higher than the periodontal disease group were selected (Table 2). The corresponding 24 proteins were subjected to three analyses of salivary proteins of 5 periodontally healthy samples and detected in all three analyses, and recorded with an average of concentrations measured three times. The fact that a protein was not detected before/after periodontitis treatment means that when a subject's saliva was analyzed three times, the corresponding protein was detected less than 2 times or not detected at all. The amino acid sequences of the protein of Table 2 can be confirmed by corresponding Uniprot IDs from UniProt (www.uniprot.org), and are represented by SEQ ID NOs: 13 to 31 of the accompanying sequence listing. The GO biological function shown in Table B indicates the function of a corresponding protein. When indicated as NA, it means that the function of a corresponding protein is not known.

TABLE B

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| O60218 | Aldo-keto | 0.040 | 0.002 | 0.014 | 5 | 4 | 5 |
| | reductase family | | | | | | |
| | 1 member B10 | | | | | | |
| Q02487 | Desmocollin-2 | 0.058 | 0.007 | 0.026 | 5 | 5 | 5 |
| Q9NP55 | BPI fold- | 0.071 | 0.011 | 0.069 | 5 | 4 | 4 |
| | containing | | | | | | |
| | family A | | | | | | |
| | member 1 | | | | | | |
| Q8NFT8 | Delta and | 0.005 | 0.001 | 0.004 | 5 | 4 | 5 |
| | Notch-like | | | | | | |
| | epidermal | | | | | | |
| | growth factor- | | | | | | |
| | related receptor | | | | | | |
| P55058 | Phospholipid | 0.040 | 0.008 | 0.037 | 5 | 3 | 4 |
| | transfer protein | | | | | | |
| P17900 | Ganglioside | 0.029 | 0.007 | 0.011 | 5 | 5 | 4 |
| | GM2 activator | | | | | | |
| A0A0A0MS15 | Ig heavy | 0.031 | 0.008 | 0.013 | 5 | 5 | 5 |
| | variable 3-49 | | | | | | |
| P18510-2 | Isoform 2 of | 0.138 | 0.036 | 0.046 | 5 | 5 | 5 |
| | Interleukin-1 | | | | | | |
| | receptor | | | | | | |
| | antagonist | | | | | | |
| | protein | | | | | | |
| Q96DR5 | BPI fold- | 0.605 | 0.163 | 0.704 | 5 | 4 | 5 |
| | containing | | | | | | |
| | family A | | | | | | |
| | member 2 | | | | | | |
| P40199 | Carcinoembryonic | 0.020 | 0.006 | 0.012 | 5 | 5 | 5 |
| | antigen- | | | | | | |
| | related cell | | | | | | |
| | adhesion | | | | | | |
| | molecule 6 | | | | | | |
| P10909-2 | Isoform 2 of | 0.038 | 0.011 | 0.037 | 5 | 5 | 5 |
| | Clusterin | | | | | | |
| P02538 | KRT6A Keratin, | 0.199 | 0.061 | 0.114 | 5 | 5 | 5 |
| | type II | | | | | | |
| | cytoskeletal 6A | | | | | | |
| P02750 | Leucine-rich | 0.036 | 0.013 | 0.027 | 5 | 5 | 5 |
| | alpha-2- | | | | | | |
| | glycoprotein | | | | | | |
| P22079 | Lactoperoxidase | 0.209 | 0.084 | 0.205 | 5 | 5 | 5 |
| A0A0G2JMB2 | Ig heavy | 1.056 | 0.460 | 0.469 | 5 | 4 | 3 |
| | constant alpha | | | | | | |
| | 2 (Fragment) | | | | | | |
| PODOY2 | Ig lambda | 4.221 | 1.850 | 3.218 | 5 | 5 | 5 |
| | constant 2 | | | | | | |
| Q9UGM3 | Deleted in | 0.036 | 0.016 | 0.026 | 5 | 5 | 5 |
| | malignant brain | | | | | | |
| | tumors 1 | | | | | | |
| | protein | | | | | | |
| P01871 | Ig heavy | 0.371 | 0.177 | 0.187 | 5 | 5 | 5 |
| | constant mu | | | | | | |
| P04406 | Glyceraldehyde- | 0.201 | 0.098 | 0.288 | 5 | 5 | 5 |
| | 3-phosphate | | | | | | |
| | dehydrogenase | | | | | | |
| A0A0J9YY99 | Ig-like domain- | 0.132 | 0.076 | 0.098 | 5 | 5 | 5 |
| | containing | | | | | | |
| | protein | | | | | | |
| P01782 | Ig heavy | 0.079 | 0.050 | 0.052 | 5 | 5 | 5 |
| | variable 3-9 | | | | | | |
| P01019 | Angiotensinogen | 0.010 | 0.006 | 0.012 | 5 | 4 | 5 |
| P02647 | Apolipoprotein | 0.247 | 0.184 | 0.204 | 5 | 4 | 5 |
| | A-I | | | | | | |
| P01008 | Antithrombin-III | 0.011 | 0.013 | 0.010 | 5 | 5 | 4 |

| | Detection count | | | | |
|---|---|---|---|---|---|
| | | Before | After | Ratio | |
| | Healthy | Treatment | Treatment | Healthy/ | GO Biological |
| Uniprot ID | group | group | group | Before | Function |
| O60218 | 15 | 6 | 13 | 18.095 | cellular detoxification of |
| | | | | | aldehyde/daunorubicin |
| | | | | | metabolic process/ |
| | | | | | doxorubicin metabolic |
| | | | | | process |
| Q02487 | 15 | 10 | 14 | 7.711 | cell adhesion/ |
| | | | | | keratinization |
| Q9NP55 | 15 | 11 | 10 | 6.552 | innate immune response |
| Q8NFT8 | 15 | 5 | 12 | 5.817 | Notch signaling pathway/ |
| | | | | | endocytosis/skeletal |
| | | | | | muscle fiber development |
| P55058 | 15 | 8 | 11 | 5.313 | ceramide transport/lipid |
| | | | | | metabolic process |
| P17900 | 15 | 10 | 10 | 4.011 | neutrophil degranulation/ |
| | | | | | ganglioside catabolic |
| | | | | | process/oligosaccharide |
| | | | | | catabolic process |
| A0A0A0MS15 | 15 | 7 | 12 | 3.821 | B cell receptor signaling |
| | | | | | pathway/innate immune |
| | | | | | response |
| P18510-2 | 15 | 13 | 13 | 3.781 | immune response |
| Q96DR5 | 15 | 11 | 14 | 3.708 | antimicrobial humoral |
| | | | | | response |
| P40199 | 15 | 11 | 14 | 3.412 | leukocyte migration/ |
| | | | | | apoptotic process/ |
| | | | | | neutrophil degranulation |
| P10909-2 | 15 | 12 | 14 | 3.404 | immune complex clearance/ |
| | | | | | innate immune response/ |
| | | | | | lipid metabolic process/ |
| | | | | | platelet degranulation |
| P02538 | 15 | 12 | 13 | 3.281 | antimicrobial humoral |
| | | | | | immune response/ |
| | | | | | keratinization/cell |
| | | | | | differentiation |
| P02750 | 15 | 14 | 13 | 2.862 | neutrophil degranulation/ |
| | | | | | positive regulation of |
| | | | | | endothelial cell |
| | | | | | proliferation |
| P22079 | 15 | 13 | 14 | 2.475 | cell redox homeostasis/ |
| | | | | | defense response to |
| | | | | | bacterium |
| A0A0G2JMB2 | 15 | 12 | 8 | 2.296 | NA |
| PODOY2 | 15 | 12 | 12 | 2.282 | innate immune response |
| Q9UGM3 | 15 | 13 | 12 | 2.240 | innate immune response/ |
| | | | | | epithelial cell |
| | | | | | differentiation |
| P01871 | 15 | 14 | 14 | 2.100 | immune response |
| P04406 | 15 | 13 | 13 | 2.050 | canonical glycolysis/ |
| | | | | | microtubule cytoskeleton |
| | | | | | organization/antimicrobial |
| | | | | | humoral immune response |
| | | | | | mediated by antimicrobial |
| | | | | | peptide |
| A0A0J9YY99 | 15 | 12 | 13 | 1.730 | innate immune response |
| P01782 | 15 | 14 | 12 | 1.584 | immune response |
| P01019 | 15 | 12 | 14 | 1.578 | cytokine secretion/aging/ |
| | | | | | blood vessel remodeling/ |
| | | | | | cell-cell signaling |
| P02647 | 15 | 10 | 12 | 1.348 | cholesterol homeostasis/ |
| | | | | | negative regulation of |
| | | | | | cytokine secretion involved |
| | | | | | in immune response/ |
| | | | | | negative regulation of |
| | | | | | interleukin-1 beta secretion/ |
| | | | | | negative regulation of |
| | | | | | inflammatory response |
| P01008 | 15 | 13 | 11 | 0.784 | blood coagulation/ |
| | | | | | cellular protein metabolic |
| | | | | | process |

The detailed detection count and amount of each protein are shown in Table B-1.

TABLE B-1

| | | Protein mol %(Individual average) | | | | |
|---|---|---|---|---|---|---|
| | Protein | Healthy group | | | | |
| Uniprot ID | description | 1 | 2 | 3 | 4 | 5 |
| PODOY2 | Ig lambda constant 2 | 3.632 | 2.545 | 1.829 | 7.390 | 5.710 |
| A0A0G2JMB2 | Ig heavy constant alpha 2 | 0.691 | 0.992 | 0.537 | 1.855 | 1.205 |
| | (Fragment) | | | | | |
| Q96DR5 | BPI fold-containing family A | 0.186 | 0.703 | 0.333 | 0.494 | 1.310 |
| | member 2 | | | | | |
| P01871 | Ig heavy constant mu | 0.205 | 0.252 | 0.183 | 0.449 | 0.765 |
| P02647 | Apolipoprotein A-I | 0.198 | 0.140 | 0.258 | 0.208 | 0.433 |
| P22079 | Lactoperoxidase | 0.091 | 0.274 | 0.166 | 0.209 | 0.305 |
| P04406 | Glyceraldehyde-3-phosphate | 0.312 | 0.120 | 0.310 | 0.197 | 0.065 |
| | dehydrogenase | | | | | |
| P02538 | KRT6A Keratin, type II cytoskeletal | 0.064 | 0.356 | 0.071 | 0.221 | 0.285 |
| | 6A | | | | | |
| P18510-2 | Isoform 2 of Interleu kin-1 receptor | 0.309 | 0.074 | 0.125 | 0.083 | 0.098 |
| | antagonist protein | | | | | |
| A0A0J9YY99 | Uncharacterized protein | 0.107 | 0.059 | 0.071 | 0.164 | 0.257 |
| | (Fragment) Ig-like domain- | | | | | |
| | containing protein | | | | | |
| P01782 | Igheavy variable 3-9 | 0.071 | 0.074 | 0.045 | 0.105 | 0.101 |
| Q9NP55 | BPI fold-containing family A | 0.052 | 0.038 | 0.039 | 0.101 | 0.123 |
| | member 1 | | | | | |
| Q02487 | Desmocollin-2 | 0.065 | 0.038 | 0.057 | 0.093 | 0.036 |
| P55058 | Phospholipid transfer protein | 0.011 | 0.055 | 0.017 | 0.013 | 0.104 |
| O60218 | Aldo-keto reductase family 1 | 0.119 | 0.009 | 0.053 | 0.007 | 0.009 |
| | member B10 | | | | | |
| P10909-2 | Isoform 2 of Clusterin | 0.016 | 0.029 | 0.015 | 0.062 | 0.069 |
| P02750 | Leucine-rich alpha-2-glycoprotein | 0.042 | 0.020 | 0.033 | 0.061 | 0.025 |
| Q9UGM3 | Deleted in malignant brain tumors | 0.020 | 0.045 | 0.019 | 0.041 | 0.052 |
| | 1 protein | | | | | |
| A0A0A0MS15 | Ig heavy variable 3-49 | 0.013 | 0.039 | 0.011 | 0.044 | 0.048 |
| P17900 | Ganglioside GM2 activator | 0.039 | 0.023 | 0.022 | 0.030 | 0.028 |
| P40199 | Carcinoembryonic antigen-related | 0.015 | 0.011 | 0.018 | 0.030 | 0.026 |
| | cell adhesion molecule 6 | | | | | |
| P01008 | Antithrombin-III | 0.010 | 0.005 | 0.012 | 0.016 | 0.010 |
| P01019 | Angiotensinogen | 0.010 | 0.007 | 0.004 | 0.016 | 0.013 |
| Q8NFT8 | Delta and Notch-like epidermal | 0.003 | 0.004 | 0.004 | 0.005 | 0.008 |
| | growth factor-related receptor | | | | | |

| | Protein mol %(Individual average) | | | | | | | | | |
|---|---|---|---|---|---|---|---|---|---|---|
| | Before Treatment group | | | | | After Treatment group | | | | |
| Uniprot ID | 1 | 2 | 3 | 4 | 5 | 1 | 2 | 3 | 4 | 5 |
| PODOY2 | 1.472 | 1.364 | 1.825 | 2.574 | 2.013 | 0.824 | 7.786 | 1.643 | 3.808 | 2.030 |
| A0A0G2JMB2 | 0.758 | 0.388 | 0.609 | 0.545 | 0.000 | 0.798 | 1.086 | 0.460 | 0.000 | 0.000 |
| Q96DR5 | 0.000 | 0.027 | 0.603 | 0.002 | 0.184 | 0.019 | 0.906 | 1.121 | 0.029 | 1.445 |
| P01871 | 0.084 | 0.044 | 0.200 | 0.046 | 0.509 | 0.142 | 0.145 | 0.144 | 0.145 | 0.357 |
| P02647 | 0.000 | 0.051 | 0.765 | 0.006 | 0.096 | 0.004 | 0.097 | 0.403 | 0.051 | 0.463 |
| P22079 | 0.093 | 0.038 | 0.178 | 0.044 | 0.070 | 0.125 | 0.234 | 0.340 | 0.078 | 0.249 |
| P04406 | 0.118 | 0.177 | 0.178 | 0.002 | 0.014 | 0.798 | 0.344 | 0.168 | 0.010 | 0.121 |
| P02538 | 0.112 | 0.037 | 0.099 | 0.040 | 0.016 | 0.084 | 0.081 | 0.039 | 0.247 | 0.117 |
| P18510-2 | 0.065 | 0.011 | 0.026 | 0.021 | 0.059 | 0.039 | 0.064 | 0.061 | 0.030 | 0.038 |
| A0A0J9YY99 | 0.045 | 0.023 | 0.093 | 0.061 | 0.157 | 0.050 | 0.125 | 0.147 | 0.072 | 0.095 |
| P01782 | 0.048 | 0.026 | 0.033 | 0.036 | 0.108 | 0.058 | 0.051 | 0.023 | 0.039 | 0.090 |
| Q9NP55 | 0.000 | 0.027 | 0.018 | 0.005 | 0.004 | 0.000 | 0.164 | 0.082 | 0.002 | 0.096 |
| Q02487 | 0.006 | 0.006 | 0.002 | 0.015 | 0.008 | 0.039 | 0.044 | 0.012 | 0.016 | 0.020 |
| P55058 | 0.004 | 0.000 | 0.021 | 0.000 | 0.012 | 0.007 | 0.041 | 0.068 | 0.000 | 0.069 |
| O60218 | 0.003 | 0.000 | 0.001 | 0.004 | 0.003 | 0.031 | 0.019 | 0.005 | 0.009 | 0.004 |
| P10909-2 | 0.003 | 0.003 | 0.024 | 0.001 | 0.025 | 0.013 | 0.050 | 0.054 | 0.011 | 0.058 |
| P02750 | 0.014 | 0.007 | 0.008 | 0.009 | 0.025 | 0.032 | 0.024 | 0.005 | 0.037 | 0.039 |
| Q9UGM3 | 0.005 | 0.009 | 0.021 | 0.012 | 0.033 | 0.006 | 0.037 | 0.024 | 0.015 | 0.047 |
| A0A0A0MS15 | 0.006 | 0.008 | 0.003 | 0.002 | 0.021 | 0.004 | 0.013 | 0.010 | 0.023 | 0.017 |
| P17900 | 0.012 | 0.001 | 0.005 | 0.002 | 0.016 | 0.017 | 0.018 | 0.006 | 0.000 | 0.015 |
| P40199 | 0.010 | 0.002 | 0.004 | 0.006 | 0.008 | 0.014 | 0.016 | 0.007 | 0.020 | 0.004 |
| P01008 | 0.003 | 0.013 | 0.004 | 0.020 | 0.029 | 0.012 | 0.008 | 0.000 | 0.028 | 0.004 |
| P01019 | 0.006 | 0.009 | 0.009 | 0.007 | 0.000 | 0.006 | 0.011 | 0.004 | 0.028 | 0.010 |
| Q8NFT8 | 0.002 | 0.000 | 0.001 | 0.000 | 0.001 | 0.003 | 0.004 | 0.005 | 0.001 | 0.006 |

In addition, among 11 proteins of Table C which were detected in all saliva samples in the periodontally healthy group and the group after the treatment of periodontitis in all of the three repeated experiments, 9 proteins having an average mol % in the healthy group 1.5-fold or higher than that of the periodontitis group were selected, and listed in order of ratios of healthy group/periodontitis group in Table 3.

The 11 proteins were subjected to three analyses of salivary proteins from 5 periodontally healthy persons and 5 persons after the treatment of periodontitis, and recorded with an average of concentrations measured three times. The fact that a protein was not detected in the periodontitis group before treatment means that, when a subject's saliva was analyzed three times, the corresponding protein was detected less than 2 times or not detected at all.

The amino acid sequences of the proteins in Table 3 may be confirmed by corresponding Uniprot IDs from UniProt (www.uniprot.org), and are represented in SEQ ID NOs: 32 to 40 of the accompanying sequence listing. The GO biological function shown in Table C indicates the function of a corresponding protein. When indicated as NA, it means that the function of a corresponding protein is not known.

TABLE C

| | | Protein mol % (Group average) | | | Number of person detected | | |
|---|---|---|---|---|---|---|---|
| | | | Before | After | | Before | After |
| | Protein | Healthy | Treatment | Treatment | Healthy | Treatment | Treatment |
| Uniprot ID | description | group | group | group | group | group | group |
| P00558 | Phosphoglycerate | 0.373 | 0.084 | 0.137 | 5 | 5 | 5 |
| | kinase 1 | | | | | | |
| Q6UWP8 | Suprabasin | 0.009 | 0.003 | 0.008 | 5 | 5 | 5 |
| Q8TDL5 | BPI fold- | 0.153 | 0.051 | 0.180 | 5 | 4 | 5 |
| | containing family | | | | | | |
| | B member 1 | | | | | | |
| Q8TAX7 | Mucin-7 | 0.021 | 0.009 | 0.020 | 5 | 5 | 5 |
| P07355-2 | Annexin A2 | 0.041 | 0.018 | 0.066 | 5 | 5 | 5 |
| P23280-2 | Carbonic | 0.302 | 0.148 | 0.294 | 5 | 5 | 5 |
| | anhydrase 6 | | | | | | |
| P35527 | Keratin, type I | 1.464 | 0.803 | 1.146 | 5 | 5 | 5 |
| | cytoskeletal 9 | | | | | | |
| P01011 | Alpha-1- | 0.035 | 0.020 | 0.043 | 5 | 4 | 5 |
| | antichymotrypsin | | | | | | |
| P01700 | Ig lambda | 0.081 | 0.052 | 0.068 | 5 | 5 | 5 |
| | variable 1-47 | | | | | | |
| P60174 | Triosephosphate | 0.395 | 0.286 | 0.374 | 5 | 5 | 5 |
| | isomerase | | | | | | |
| P04083 | Annexin A1 | 0.070 | 0.057 | 0.119 | 5 | 5 | 5 |

| | Detection count | | | | |
|---|---|---|---|---|---|
| | | Before | After | Ratio | |
| | Healthy | Treatment | Treatment | Healthy/ | GO Biological |
| Uniprot ID | group | group | group | Before | Function |
| P00558 | 15 | 14 | 15 | 4.452 | canonical glycolysis |
| Q6UWP8 | 15 | 13 | 15 | 3.197 | NA |
| Q8TDL5 | 15 | 11 | 15 | 3.021 | antimicrobial humoral |
| | | | | | response |
| Q8TAX7 | 15 | 14 | 15 | 2.385 | antimicrobial humoral |
| | | | | | immune response |
| P07355-2 | 15 | 12 | 15 | 2.304 | angiogenesis/ |
| | | | | | osteoclast |
| | | | | | development/ |
| | | | | | neutrophil |
| | | | | | degranulation |
| P23280-2 | 15 | 14 | 15 | 2.041 | bicarbonate transport |
| P35527 | 15 | 14 | 15 | 1.822 | keratinization |
| P01011 | 15 | 12 | 15 | 1.758 | acute-phase response/ |
| | | | | | inflammatory response |
| P01700 | 15 | 14 | 15 | 1.554 | immune response |
| P60174 | 15 | 14 | 15 | 1.384 | canonical glycolysis |
| P04083 | 15 | 14 | 15 | 1.224 | actin cytoskeleton |
| | | | | | reorganization/ |
| | | | | | neutrophil clearance |

The detailed detection count and amount of each protein are shown in Table C-1.

TABLE C-1

| | | Protein mol % (individual person average) | | | | | | | | | | | | | | |
|---|---|---|---|---|---|---|---|---|---|---|---|---|---|---|---|---|
| | Protein | Healthy group | | | | | Before Treatment group | | | | | After Treatment group | | | | |
| Uniprot ID | description | 1 | 2 | 3 | 4 | 5 | 1 | 2 | 3 | 4 | 5 | 1 | 2 | 3 | 4 | 5 |
| P35527 | Keratin, type I | 0.574 | 3.260 | 0.630 | 1.901 | 0.953 | 0.810 | 0.523 | 1.115 | 0.816 | 0.753 | 0.555 | 1.087 | 0.965 | 1.926 | 1.196 |
| | cytoskeletal 9 | | | | | | | | | | | | | | | |
| P60174 | Triosephosphate isomerase | 0.693 | 0.255 | 0.641 | 0.223 | 0.166 | 0.419 | 0.147 | 0.271 | 0.175 | 0.416 | 0.556 | 0.411 | 0.250 | 0.366 | 0.285 |
| P00558 | Phosphoglycerate kinase 1 | 1.080 | 0.176 | 0.508 | 0.051 | 0.049 | 0.243 | 0.097 | 0.066 | 0.010 | 0.003 | 0.297 | 0.235 | 0.027 | 0.011 | 0.117 |
| P23280-2 | Carbonic anhydrase 6 | 0.103 | 0.447 | 0.251 | 0.287 | 0.423 | 0.147 | 0.075 | 0.248 | 0.096 | 0.175 | 0.213 | 0.308 | 0.466 | 0.157 | 0.327 |
| Q8TDL5 | BPI fold-containing | 0.252 | 0.042 | 0.092 | 0.208 | 0.171 | 0.038 | 0.155 | 0.007 | 0.054 | 0.000 | 0.102 | 0.621 | 0.084 | 0.036 | 0.060 |
| | family B member 1 | | | | | | | | | | | | | | | |
| P01700 | Ig lambda variable 1047 | 0.069 | 0.063 | 0.065 | 0.110 | 0.096 | 0.028 | 0.023 | 0.026 | 0.052 | 0.130 | 0.031 | 0.073 | 0.046 | 0.066 | 0.125 |
| P04083 | Annexin A1 | 0.084 | 0.015 | 0.139 | 0.049 | 0.063 | 0.114 | 0.036 | 0.092 | 0.029 | 0.015 | 0.253 | 0.162 | 0.107 | 0.056 | 0.018 |
| P07355-2 | Annexin A2 | 0.045 | 0.049 | 0.040 | 0.040 | 0.034 | 0.034 | 0.007 | 0.025 | 0.007 | 0.016 | 0.063 | 0.075 | 0.081 | 0.065 | 0.045 |
| P01011 | Alpha-1-antichymotrypsin | 0.016 | 0.023 | 0.010 | 0.088 | 0.036 | 0.024 | 0.027 | 0.000 | 0.019 | 0.030 | 0.038 | 0.044 | 0.042 | 0.030 | 0.062 |
| Q8TAX7 | Mucin-7 | 0.010 | 0.026 | 0.022 | 0.020 | 0.028 | 0.013 | 0.004 | 0.010 | 0.003 | 0.015 | 0.015 | 0.025 | 0.017 | 0.019 | 0.025 |
| Q6UWP8 | Suprabasin | 0.006 | 0.010 | 0.009 | 0.013 | 0.008 | 0.003 | 0.002 | 0.004 | 0.002 | 0.004 | 0.004 | 0.007 | 0.007 | 0.012 | 0.008 |

Finally, among the proteins detected in all saliva samples in the periodontally healthy group and the periodontitis groups before and after treatment, three proteins, which had low concentrations before treatment, but are increased in concentration 2-fold or higher after treatment of periodontitis, and in the periodontally healthy group, are also increased in concentration 2-fold or higher than those before treatment, are shown in Tables D and 4. The corresponding three proteins were subjected to three repeated analyses of salivary proteins from 5 periodontally healthy persons and 5 patients each with periodontitis before and after treatment and detected in all three analyses, and recorded with an average of concentrations measured three times. The ratios in the rightmost column represent a concentration ratio after periodontitis treatment with respect to a concentration of the periodontitis-treated group and a concentration ratio of the healthy group with respect to a concentration before periodontitis treatment. Compared with before periodontitis treatment, a 2- to 4-fold or larger difference is shown after periodontitis treatment and in healthy gums.

The amino acid sequences of the proteins in Tables D and 4 may be confirmed by corresponding Uniprot IDs from UniProt (www.uniprot.org), and represented by SEQ ID NOs: 41 to 43 of the accompanying sequence listing. The GO biological function shown in Table D indicates the function of a corresponding protein. When indicated as NA, it means that the function of a corresponding protein is not known.

TABLE D

| | | Protein mol % (Group average) | | | Number of person detected | |
|---|---|---|---|---|---|---|
| | | | Before | After | | Before |
| | Protein | Healthy | Treatment | Treatment | Healthy | Treatment |
| Uniprot ID | description | group | group | group | group | group |
| P30086 | Phosphatidylethanolamine- | 0.204 | 0.049 | 0.149 | 5 | 5 |
| | binding protein 1 | | | | | |
| Q02413 | Desmoglein-1 | 0.024 | 0.007 | 0.022 | 5 | 5 |
| P25311 | Zinc-alpha-2- | 2.057 | 1.025 | 2.141 | 5 | 5 |
| | glycoprotein | | | | | |

| | Number of person detected | Detection count | | | | |
|---|---|---|---|---|---|---|
| | After | | Before | After | Ratio | |
| | Treatment | Healthy | Treatment | Treatment | Healthy/ | GO Biological |
| Uniprot ID | group | group | group | group | Before | Function |
| P30086 | 5 | 15 | 15 | 15 | 4.207 | MAPK |
| | | | | | | cascade |
| Q02413 | 5 | 15 | 15 | 15 | 3.583 | cell-cell |
| | | | | | | adhesion/ |
| | | | | | | keratinization |
| P25311 | 5 | 15 | 15 | 15 | 2.007 | cell |
| | | | | | | adhesion/ |
| | | | | | | immune |
| | | | | | | response |

The detailed detection frequency and amount of each protein are shown in Table D-1.

TABLE D-1

| | | Protein mol % (individual person average) | | | | | | | | | | | | | | |
|---|---|---|---|---|---|---|---|---|---|---|---|---|---|---|---|---|
| | Protein | Healthy group | | | | | Before Treatment group | | | | | After Treatment group | | | | |
| Uniprot ID | description | 1 | 2 | 3 | 4 | 5 | 1 | 2 | 3 | 4 | 5 | 1 | 2 | 3 | 4 | 5 |
| P25311 | Zinc-alpha-2-glycoprotein | 1.610 | 2.010 | 1.514 | 2.642 | 2.508 | 1.066 | 0.680 | 1.203 | 0.424 | 1.750 | 1.921 | 2.301 | 1.881 | 1.679 | 2.922 |
| P30086 | Phosphatidylethanolamine- | 0.319 | 0.136 | 0.242 | 0.138 | 0.185 | 0.106 | 0.025 | 0.045 | 0.037 | 0.029 | 0.163 | 0.229 | 0.116 | 0.080 | 0.159 |
| | binding protein 1 | | | | | | | | | | | | | | | |
| Q02413 | Desmoglein-1 | 0.036 | 0.017 | 0.023 | 0.028 | 0.014 | 0.009 | 0.004 | 0.009 | 0.004 | 0.007 | 0.023 | 0.028 | 0.021 | 0.022 | 0.016 |

Although embodiments of the present invention have been described above, it should be understood by those of ordinary skill in the art that the present invention is not limited to the embodiments disclosed above, and may be embodied in many different forms, the embodiments disclosed herein can be easily modified into other specific forms without departing from the technical spirit or essential features of the present invention. Therefore, the embodiments described above should be interpreted as illustrative and not limited in any aspect.

1. A method of detecting a marker protein for diagnosing a periodontal condition to provide information required for diagnosis of a periodontal condition, the method comprising:
i) detecting one or more proteins selected from the group consisting of Hemoglobin subunit delta, Histone H3.1, Neutrophil collagenase, Myosin-9, WD repeat-containing protein 1, Cathepsin G, Serpin B10, Vimentin, Protein S100-P, Heme-binding protein 2, Alpha-actinin-4, Protein disulfide-isomerase, Ig lambda constant 2, Ig heavy constant alpha 2, BPI fold-containing family A member 2, Ig heavy constant mu, Lactoperoxidase, Glyceraldehyde-3-phosphate dehydrogenase, KRT6A Keratin, type II cytoskeletal 6A, Isoform 2 of Interleukin-1 receptor antagonist protein, BPI fold-containing family A member 1, Desmocollin-2, Phospholipid transfer protein, Aldo-keto reductase family 1 member B10, Isoform 2 of Clusterin, Leucine-rich alpha-2-glycoprotein, Deleted in malignant brain tumors 1 protein, Ig heavy variable 3-49, Ganglioside GM2 activator, Carcinoembryonic antigen-related cell adhesion molecule 6, Delta and Notch-like epidermal growth factor-related receptor, Phosphoglycerate kinase 1, Suprabasin, BPI fold-containing family B member 1, Mucin-7, Annexin A2, Carbonic anhydrase 6, Keratin, type I cytoskeletal 9, Alpha-1-antichymotrypsin, Ig lambda variable 1-47, Zinc-alpha-2-glycoprotein, Desmoglein-1 and Phosphatidylethanolamine-binding protein 1 from a subject's sample; and
ii) associating the subject with the diagnosis of a periodontal condition, if the above-listed one or more proteins are increased or decreased in concentration in comparison with a control sample.

2. The method of claim 1, wherein the subject is determined as having an abnormal periodontal condition, if one or more proteins selected from the group consisting of Hemoglobin subunit delta, Histone H3.1, Neutrophil collagenase, Myosin-9, WD repeat-containing protein 1, Cathepsin G, Serpin B10, Vimentin and Protein S100-P increase in concentration compared with a normal control sample, and/or if one or more proteins selected from the group consisting of Heme-binding protein 2, Alpha-actinin-4, Protein disulfide-isomerase, Ig lambda constant 2, Ig heavy constant alpha 2, BPI fold-containing family A member 2, Ig heavy constant mu, Lactoperoxidase, Glyceraldehyde-3-phosphate dehydrogenase, KRT6A Keratin, type II cytoskeletal 6A, Isoform 2 of Interleukin-1 receptor antagonist protein, BPI fold-containing family A member 1, Desmocollin-2, Phospholipid transfer protein, Aldo-keto reductase family 1 member B10, Isoform 2 of Clusterin, Leucine-rich alpha-2-glycoprotein, Deleted in malignant brain tumors 1 protein, Ig heavy variable 3-49, Ganglioside GM2 activator, Carcinoembryonic antigen-related cell adhesion molecule 6, Delta and Notch-like epidermal growth factor-related receptor, Phosphoglycerate kinase 1, Suprabasin, BPI fold-containing family B member 1, Mucin-7, Annexin A2, Carbonic anhydrase 6, Keratin, type I cytoskeletal 9, Alpha-1-antichymotrypsin, Ig lambda variable 1-47, Zinc-alpha-2-glycoprotein, Desmoglein-1 and Phosphatidylethanolamine-binding protein 1 decrease in concentration compared with a normal control sample.

3. The method of claim 1, wherein the subject is determined as having a normal periodontal condition, if one or more proteins selected from the group consisting of Hemoglobin subunit delta, Histone H3.1, Neutrophil collagenase, Myosin-9, WD repeat-containing protein 1, Cathepsin G, Serpin B10, Vimentin and Protein S100-P decrease in concentration compared with an abnormal control sample, and/or if one or more proteins selected from the group consisting of Heme-binding protein 2, Alpha-actinin-4, Protein disulfide-isomerase, Ig lambda constant 2, Ig heavy constant alpha 2, BPI fold-containing family A member 2, Ig heavy constant mu, Lactoperoxidase, Glyceraldehyde-3-phosphate dehydrogenase, KRT6A Keratin, type II cytoskeletal 6A, Isoform 2 of Interleukin-1 receptor antagonist protein, BPI fold-containing family A member 1, Desmocollin-2, Phospholipid transfer protein, Aldo-keto reductase family 1 member B10, Isoform 2 of Clusterin, Leucine-rich alpha-2-glycoprotein, Deleted in malignant brain tumors 1 protein, Ig heavy variable 3-49, Ganglioside GM2 activator, Carcinoembryonic antigen-related cell adhesion molecule 6, Delta and Notch-like epidermal growth factor-related receptor, Phosphoglycerate kinase 1, Suprabasin, BPI fold-containing family B member 1, Mucin-7, Annexin A2, Carbonic anhydrase 6, Keratin, type I cytoskeletal 9, Alpha-1-antichymotrypsin, Ig lambda variable 1-47, Zinc-alpha-2-glycoprotein, Desmoglein-1 and Phosphatidylethanolamine-binding protein 1 increase in concentration compared with an abnormal control sample.

4. The method of claim 1, wherein the detection is performed by a method of detecting an antigen-antibody complex.

5. A composition for diagnosing a periodontal condition, comprising a reagent for detecting one or more proteins listed in claim 1 or an immunogenic fragment thereof.

6. The composition of claim 5, wherein the reagent is an antibody, a substrate, an aptamer, an avimer, a peptidomimetic, a receptor or a ligand, which are specific for each protein or a fragment thereof.

7. A kit for diagnosing a periodontal condition, comprising the composition of claim 5.

8. The method of claim 2, wherein the detection is performed by a method of detecting an antigen-antibody complex.

9. The method of claim 3, wherein the detection is performed by a method of detecting an antigen-antibody complex.
